(19) United States
(12) Patent Application Publication
(10) Pub. No.: US 2009/0036856 A1
(43) Pub. Date: Feb. 5, 2009

(54) TRIGGERABLE SELF-GENERATING LIQUID FOAM BARRIER/INTERCEPTOR

(75) Inventors: Garry Roland Woltman, Appleton, WI (US); David William Koenig, Menasha, WI (US); Mary Lou McDaniel, Appleton, WI (US); Carrie Pateras, Boardman, OH (US)

Correspondence Address: Christopher M. Goff (27839);ARMSTRONG TEASDALE LLP, ONE METROPOLITAN SQUARE, SUITE 2600, ST. LOUIS, MO, 63102, US

(73) Assignee: KIMBERLY-CLARK WORLDWIDE, INC., Neenah, WI (US)

(21) Appl. No.: 11/831,598

(22) Filed: Jul. 31, 2007

(51) Int. Cl. A61F 13/15

(52) U.S. Cl. 604/385.01; 604/385.04; 604/385.101

(57) ABSTRACT

The present disclosure generally relates to absorbent articles, such as diapers, training pants, swim pants, incontinence articles, feminine care articles, and the like, that comprise a system for generating a fluid or odor barrier or interceptor. More specifically, the articles comprise a pre-use foam composition that when contacted with a triggering agent, expands to produce a foam barrier or interceptor structure that provides protection against leakage of the absorbent article.

### BACKGROUND OF DISCLOSURE

The present disclosure generally relates to absorbent articles, such as diapers, training pants, swim pants, incontinence articles, feminine care articles, and the like, that comprise a system for generating a fluid or odor barrier or interceptor. More specifically, the articles comprise a pre-use foam composition that when contacted with a triggering agent, expands to produce a foam barrier or interceptor structure. The foam barrier or interceptor structure provides protection against leakage of the absorbent article, and may act to absorb exudates.

A large variety of absorbent articles are known, such as diapers, training pants, swim pants, incontinence articles, feminine care articles, and the like. Such absorbent articles commonly include a liquid-permeable, bodyfacing topsheet, an absorbent core, and a liquid-impermeable outer cover.

The major function of absorbent articles is to absorb and contain body exudates. Such articles are thus intended to prevent body exudates from soiling, wetting, or otherwise contaminating clothing or other articles, such as bedding, that come in contact with the wearer. The most common mode of failure for such products is the leakage of fluids when using the articles, particularly from around the side edges of the article, or out of the gaps between the article and the wearer's leg or waist, in the case of diapers or training pants.

Leakage of the exuded body fluids from the absorbent article typically occurs once the article becomes saturated with absorbed exudate. However, in practice, premature leakage of exudate can also often occur before saturation of the article is reached. Leakage causes staining of the support or associated garments and therefore frequent changing of the absorbent article is often necessary to prevent such staining. Premature leakage can be caused by exudate never coming in contact with or remaining on the top layers of absorbent material at a central area of the absorbent article, which then causes the exudate to migrate by wicking or flowing across the upper surface of the article from the central area to the side edges thereof, typically the longitudinal side edges, but also the transverse side edges, before it has penetrated and/or fully absorbed within the main bulk of the absorbent core.

One approach used to address the problem of exudate leakage has been to increase the overall coverage area of the absorbent article, e.g., by increasing the surface area of the undergarment the product covers. This approach, however, requires larger product size, which can result in increased discomfort to the user and a lack of product discretion.

Various attempts have also been made to incorporate structures in absorbent articles to reduce or prevent premature leakage, including ready-made embossed walls or channels, polymeric or other preformed liquid impermeable barrier walls, and the like. However such attempts have not been completely successful at eliminating the premature leakage problem. Certain proposed solutions may even exacerbate the problem. For example, polymeric film barriers have been used along the side edges of feminine pads on each lateral side of a central insult area. However, due to any number of factors including slippage of the product in use, relatively heavy menstrual flow, etc. there may often be times when menstrual fluid impacts or insults the pad or the film barriers. The fluid cannot permeate through the polymeric film barrier and is directed either towards the inboard insult area or to the side edges of the article thus resulting in leakage. Also the fluid smears across the film barrier resulting in significant and potentially embarrassing staining of the article.

Another method of preventing side leakage has been to extend wings from the edges of a sanitary napkin. The wings generally extend over the edges of the undergarment crotch portion and adhere to the underside of the crotch portion or to themselves. The wing is designed to conform to the undergarment and not to the wearer's body. This lack of body conformance limits effectiveness at preventing side leakage. Furthermore, the conformance of the wings to the undergarment can actually contribute to side leakage. For instance, it is possible that these elasticized edges or wings will fold inward, partially occluding the cover surface and thereby diminishing the efficacy of the sanitary napkin. In some cases this folding results in the edges actually contributing to the incidence of failure.

Additionally, elasticized leg flaps or elastic side gathers have been incorporated into disposable diapers and training pants, and sanitary napkins have been constructed having elasticized sides that urge the sides upward or cause the sanitary napkin to form a cup shape. The elasticized sides generally form a “bucket” above the topsheet of the absorbent article to capture any free exudate, which is later absorbed by the absorbent article. Elasticized leg flaps are generally formed from an elastic member being enclosed in the continuous topsheet and outer cover which extend beyond the edges of the absorbent core. These elasticized leg flaps act to prevent wicking and overflow from the fluid laden diaper or napkin to clothing contacting the edges of the absorbent article in that the elasticized leg flaps present a fluid impervious barrier between the edge of the diaper and the contacting clothing, and in addition, provide a gasketing action about the legs of the wearer. However, leakage along the perimeter of the diaper or sanitary napkin may still occur. As liquids are discharged onto the topsheet, some of the liquid flows on the surface of the topsheet and some of the liquid is absorbed by and wicks through the topsheet and through gaps between the elasticized leg flaps and side gathers and the wearer's body. As the liquid migrates toward the edges of the diaper through these gaps, it may come in contact with clothing or undergarments where it can be absorbed by and wicked into such garments.

Still a further approach to the problem of leakage, and more typically of side leakage in absorbent articles, is the provision of physical barriers on the body facing surface of the absorbent article, such as raised elements, or alternatively barrier cuffs or walls which are capable of standing upwards from the body facing surface of the absorbent article during wear. The physical barriers restrain the free flow of body exudates on the topsheet of an absorbent article, containing the exudates within the article. There are, however, several drawbacks to physical barrier structures. For instance, it has been found that liquid can wick underneath and beyond the barrier cuffs and thus beyond the elasticized leg flaps of diapers, training pants, or sanitary napkins, and soil the wearer's clothing because the diaper construction does not present a barrier to the wicking of liquid through the topsheet. Physical barrier structures can also be rather cumbersome, and the comfort of articles comprising physical barrier structures is also an issue. Additionally, barriers tend to push the absorbent product away from the body of a wearer when a portion of the barrier comes in contact with the body, resulting in poor conformity between the article and the wearer's body.

Some attempts have been made to prevent leakage by improving the ability of absorbent products to contour to the wearer's body. For instance, U.S. Patent App. Publ. No. 2004/0116883 discloses absorbent articles comprising a body conformance system comprising substantially free-flowing particulate material constrained by a flexible containment layer. The use of the free-flowing particulate material allows for better conformance and fit of the absorbent article to the wearer's body. The ability of this article to conform to the body of the wearer, especially within the folds of the crotch region, is however, still limited by the lack of conformability of the containment layer. As a result, gaps between the article and the user's body may still be present.

There is thus a clear need for absorbent articles that comprise fluid or odor barriers or interceptors that are comfortable, and that also conform to a user's body, providing a better fit and preventing or reducing premature leakage of body exudates from the article.

### SUMMARY OF THE DISCLOSURE

The present disclosure generally relates to absorbent articles, such as diapers, training pants, swim pants, incontinence articles, feminine care articles, and the like, that comprise a system for generating a fluid or odor barrier or interceptor. More specifically, the articles comprise a pre-use foam composition that when contacted with a triggering agent, expands to produce a foam barrier and/or interceptor structure. The foam barrier or interceptor structure provides protection against leakage of the absorbent article.

In one aspect, the present disclosure is directed to an absorbent article comprising a topsheet; an absorbent core disposed beneath the topsheet; and a system for generating a stable foam structure on a body facing surface of the topsheet, the system comprising a pre-use foam composition comprising a gas providing agent and a foam forming agent, wherein the pre-use foam composition is disposed on the absorbent article in a manner such that when the gas providing agent is contacted with a triggering agent, the gas providing agent releases a gas that combines with the foam forming agent to form the stable foam structure, wherein the stable foam structure acts as a fluid barrier against the flow of a body exudate across the barrier, as an interceptor, or as a fluid barrier and an interceptor.

In another aspect, the present disclosure is directed to a composition for generating a stable foam structure, the composition comprising a gas providing agent and a foam forming agent, wherein the gas providing agent releases a gas that combines with the foam forming agent to form the stable foam structure when the composition is contacted with a triggering agent.

Other objects and features will be in part apparent and in part pointed out hereinafter.

### BRIEF DESCRIPTION OF THE DRAWINGS

FIG. 1 is a perspective and partial cut-away of an absorbent article according to the disclosure.

FIG. 2 is a cross-sectional view of the absorbent article taken along the lines indicated in FIG. 1.

FIG. 3 is a top view of an alternate embodiment of an absorbent article according to the disclosure.

FIG. 4 is a top plan view of an alternate embodiment of the disclosure.

FIG. 5 is a top plan view of an alternate embodiment of the disclosure.

FIG. 6 is a top plan view of an alternate embodiment of the disclosure.

FIG. 7 is a top plan view of an alternate embodiment of the disclosure.

FIG. 8 is a cross-sectional view of an absorbent article of the disclosure.

FIG. 9 is a top plan of a feminine napkin of the present disclosure having wings.

FIG. 10 is a bottom plan of the napkin shown in FIG. 9.

FIG. 11 is a section taken in the plane of line 11-11 of FIG. 9.

FIG. 12 representatively shows a partially cut away top plan view of an absorbent article in a stretched and laid flat condition with the surface of the article which contacts the skin of the wearer facing the viewer.

FIG. 13 representatively shows a sectional view of the absorbent article of FIG. 12 taken along line 13-13.

FIG. 14 is plan view of an absorbent article of the present disclosure illustrated in the form of a diaper shown unfastened and laid flat, with the surface that faces a wearer facing up.

Corresponding reference characters indicate corresponding parts throughout the drawings.

### DETAILED DESCRIPTION OF THE DISCLOSURE

The present disclosure generally relates to absorbent articles, such as diapers, training pants, swim pants, incontinence articles, feminine care articles, and the like. The absorbent articles generally comprise a topsheet (also referred to herein as a bodyside liner), an absorbent core disposed beneath the topsheet, and a system for generating a barrier and/or interceptor structure on the body facing surface of the topsheet. More particularly, the system comprises a pre-use foam composition that, when contacted with a triggering agent, expands to produce a foam structure that acts as a fluid and/or odor barrier against the flow of bodily exudates across the barrier, or as a fluid interceptor.

One of the problems associated with currently available barrier/interceptor structures is the inability of the structure to adequately prevent premature leakage. Oftentimes leakage results from the inability of the barrier/interceptor structure to adequately prevent body exudates from passing through gaps located between the top of the absorbent article and the wearer's body. Obtaining a barrier/interceptor structure that has adequate gap filling capability is particularly problematic given the individualistic nature of a wearer's body. Not only are the size, shape, and topology of each wearer's body unique, but the topology of the wearer's crotch region is also constantly changing as the wearer moves. Furthermore, crotch regions have many intricate contours that make it difficult to adequately fill gaps between the body and the absorbent article.

To illustrate the intricacy of the crotch region to which the barrier/interceptor structures need to conform and corresponding created spaces where they need to fit in order to prevent leakage, it will be helpful to describe the form of a wearer's body, for example a woman's body. The general overall body form and the created spaces are the combination of the form and created spaces created by the individual anatomical structures. So, the description begins with description of the individual structures.

A general description of the anatomical structures can be found in The Illustrated Running Press Edition of the American Classic Gray's Anatomy (1974) by Henry Gray and Structure and Function in Man (1974) by Stanley W. Jacob, M.D., F.A.C.S, and relevant portions are included herein by reference. The general form can be found in Anatomy for an Artist: Elements of Form by Eliot Goldfinger and relevant portions are included herein by reference. The general description of the pubic hair covering these regions can be found in Woman's Body: A Manual for Life and relevant portions are included herein by reference.

The female anatomical structures to be described include the leg and the lower torso. The external anatomical structures of the lower torso include gluteal region and perineum region. The gluteal region includes the buttocks and the anus. The anatomical structure involved on the leg is the medial surface of the upper thigh.

The gluteal region includes generally the buttocks and anus and is typically bound in front by the line of the buttocks and the gluteal folds, in the back by the sacral triangle and the sides by lines extending through the greater trochanters. The shape of the gluteal region is roughly hemispherical and convex, and is determined by a series of muscles including the gluteus maximus and a series of fat pads including the posterior gluteal fat pad. The line of the buttocks separates the gluteal region and the perineum region.

The upper thigh region includes typically the right and left thigh and is typically bound on top by the thigh lines and the sides by the front and back of the leg. The thigh lines are two lines that are on either side of the labia and each of the lines runs along the line of the inguinal ligment to the gluteal folds and marks where the upper thigh meets the lower torso. The shape of the region is roughly a portion of a tapered cylinder and convex, and is shaped by a series of muscle groups including the gracilis, pectineus, adductor longus, adductor brevis, and adductor magnus and series of fat pads including the inner thigh fat pad.

The perineum region, which extends from the inferior outlet of the pelvis to the bony structure of the coccyx, is comprised of two divisions, the urogenital triangle and the anal division or obstetrical perineum. The region includes the external organs of reproduction; the mons pubis, labia majora and minora, clitoris, meatus urinarius and the opening to the vagina. The region is generally bound in front by the lower abdominal line, on the sides the thigh lines, and in the back the line of the buttocks. The abdominal line is a line that passes across the top of the pubis. The lines of the buttocks are lines that connect the thigh lines to the gluteal cleft. For convenience in describing the form and created spaces in the perineum region, this region will be subdivided into three regions including an anterior region including the mons pubis, a central region including the labia majora and minora, and posterior region. The anterior region is bound in front by the lower abdominal line, in back by anterior commissure, and on the sides by line of the labia. The central region is bound in front by the anterior commissure, in the back by the posterior commissure, and on the side by the line of the labia. The posterior region is bound in front by the line of the labia, in the back by the lines of the buttocks, and on the sides the thigh line.

The vulvar region includes the female external genitalia and generally includes the anterior and central regions of the perineum. The mons pubis [or veneris] is generally a rounded eminence in front of the symphysis pubis, formed by a collection of fatty tissue including the pubic fat pad beneath the integument and is generally covered with pubic hair. The labia majora are generally two prominent longitudinal cutaneous folds extending downward from the mons veneris to the anterior boundary of the perineum, and generally enclosing the common urinary-sexual opening. The space between the two folds is the labial cleft. Each labium has generally two surfaces, an outer, which is pigmented and covered generally with strong, crisp pubic hairs, and an inner within the labia cleft, which is smooth and is beset with large sebaceous follicles and is continuous with the genito-urinary mucous tract; between the two there is considerable quantity of areolar tissue, fat including the labia fat pad, and tissue besides vessels, meeting the anterior commissure. Posteriorly they are typically not joined, but generally appear to become lost in the neighboring integument, terminating close to, and nearly parallel with each other. Together with the connecting skin between them, they form the posterior commissure or posterior boundary of the vulval orifice. The interval between the posterior commissure and the anus constitutes the perineum region. The fourchette is the anterior edge of the perineum, and between it and the hymen is a depression, the fossa navicularis. The line of the labia separates the labia and the perineum region.

The labia minora are two small cutaneous folds, situated generally within the labia majora, and extending from the clitoris obliquely downward, outward, and backward on each side of the orifice of the vagina, between which and the labia majora they are lost. Anteriorly, the two labia minora meet and form the fraenum of the clitoris. The prepuce of the clitoris, passing backward on each side, is inserted, as it were, into the labium minora, but is not actually a part of them. The labia minor are really modified skin. Their internal surfaces have numerous sebaceous follicles. The labia minora generally conceal the clitoris, vestibule, the orifice of the urethra (meatus urinarius), the orifice of the vagina, Glands of Bartholin, and the Bulbi Vestibuli. The clitoris in an erectile structure situated beneath the anterior commissure, partially hidden between the anterior extremities of the labia minora. The vestibule generally is a triangular smooth surface that lies between the clitoris and entrance of the vagina is a triangular smooth surface and is bound on each side by the labia minora. The orifice of the urethra (meatus urinarius) is situated at the back part of the vestibule, about an inch below the clitoris and near the margin of the vagina, surrounded by a prominent elevation of the mucous membrane. Below the meatus urinarius is the orifice of the vagina, more or less closed in the virgin by a membranous fold, the hymen. Glands of bartholin are on each side of the commencement of the vagina, and behind the hymen. Bulbi Vestibuli extends from the clitoris along either side of the vestibule and lies a little behind the labia minora.

The form of the perineum, gluteal, and upper thigh regions combine to form a very intricate skin topography and spaces. The roughly two-hemispherical-like forms of the buttocks, the roughly tapered-cylinder-like form of the upper thigh, split-teardrop-like form of the vulvar region create intricate generally convex topography with intersections to form a series of recesses. The generally convex topography of the buttocks, the vulvar region, and upper thigh join to create spaces including two inner thigh grooves along two thigh lines, a depression in the posterior perineum region and a cleft extending through the labia and gluteal clefts. The grooves, depression, and cleft are like interconnected recesses in the topography. The central region general has lateral sides separated by a distal surface created by the labial cleft and includes the labial cleft.

Pubic hair generally cover some of these regions and fill in a portion of these recesses especially the labial cleft and the portion of the groove of the thigh parallel to the labial cleft to create a hair surface topography. The hair topography is the surface topography of an imaginary distal surface created by the hair. The depression of the perineum, thigh groove parallel to the gluteal cleft, and the gluteal cleft generally has little or no pubic hair. The skin topography combines with the hair topography to create an overall body topography.

This intricate space created by the intricate body form in this region of the body varies between women in both size and form, and varies with the position and movement of the women. Some of these variations are summarized in “Female genital appearance: ‘normality’ unfolds” by Jillian Lloyd et. al., BJOG: An International Journal of Obstetrics and Gynecology, May 2005, Vol. 112, pp. 643-646 and is included herein by reference.

As can be seen from the above discussion, the crotch region is very intricate and contains numerous contours and gaps through which exudates may pass, making it difficult for currently available barrier/interceptor structures to fill gaps between the body and the absorbent article, and thus fully protect against absorbent article leakage.

For instance, prior efforts to prevent leakage have focused largely on incorporating some type of physical barrier/interceptor structure, such as polymeric or other liquid impermeable barrier cuffs or walls, elasticized leg flaps or elastic side gathers, and the like, into absorbent articles. Such structures, however, are typically pre-formed, with a predetermined size and shape, and are therefore incapable of being adjusted to take into account the individual nature of a wearer's body, or of maintaining conformance with the intricate and changing topology of the wearer's body. As a result, these structures fail to adequately fill gaps between the wearer's body and the absorbent article through which exudates may pass, resulting in leakage of the absorbent article. Additionally, the inflexible nature of the structures may produce irritating pressure-points against the wearer's body, resulting in an uncomfortable product. The inclusion of physical barrier/interceptor structures in absorbent products also increases the overall product size and bulk, which is disadvantageous in terms of comfort, product discretion, and packaging concerns.

The present disclosure addresses these issues by providing absorbent articles comprising a system for generating a barrier and/or interceptor structure on the body facing surface of the topsheet. The system comprises a pre-use foam composition that, when contacted with a triggering agent, expands to produce a foam structure that acts as a fluid and/or odor barrier against the flow of bodily exudates across the barrier, and/or as a fluid interceptor. Advantageously, the foam barrier/interceptor structure of the present disclosure is not a pre-formed structure, and is not constrained within any containment layer or envelope. Rather, it is formed on the body-facing surface of the absorbent article when the pre-use foam composition is contacted by the triggering agent. As a result, the gap-filling ability of the foam barrier/interceptor structure is not constrained by the conformability of a containment device or envelope. Additionally, the formed structure is flexible, capable of being adapted to the different topologies of a wearer's crotch region, and capable of maintaining a good seal between the absorbent article and the wearer's body, even during wearer movement. Furthermore, because the barrier/interceptor structure is not formed until the pre-use foam composition is contacted by the triggering agent, bulky or cumbersome pre-formed barrier/interceptor structures are not required, and absorbent articles may be provided that have a low pre-triggering bulk and improved comfort when worn.

The foam structure produced when the pre-use foam composition contacts a triggering agent can act as a fluid and/or odor barrier, an interceptor, or as both a barrier and an interceptor, depending on the location and desired function of the foam structure. For instance, the structure may act to capture or block the leakage of any fluid or bodily exudate that is not initially absorbed by the absorbent article. Alternately, the structure may act as a foam interceptor, acquiring or intercepting fluids that are on the body of the user. In certain embodiments, the foam structure may act as both a barrier and an interceptor. In either case, the foam produced from contact of the pre-use foam composition with the triggering agent expands to fill the void between the absorbent article and the user's body, thus creating a comfortable foam barrier and/or interceptor that conforms to the user's body and acts to reduce or prevent leakage of the absorbent article. Alternately, the foam need not expand to fill the entire void, but rather, may expand sufficiently to form a raised barrier structure on the bodyfacing surface of the topsheet.

As noted above, the foam barrier/interceptor structure is formed when a triggering agent contacts a pre-use foam composition present in the absorbent article. The pre-use foam composition comprises a gas providing agent that releases a gas upon contact with the triggering agent and a foam forming agent that combines with the released gas to form the foam barrier and/or interceptor structure. Optionally, the pre-use foam composition may further comprise a foam forming adjuvant that improves the properties of the foam formed when the triggering agent contacts the pre-use foam composition. For instance, the foam forming adjuvant may comprise a foam stabilizing agent that stabilizes the foam barrier/interceptor structure, a skin feel enhancing agent, or other agents that improve the properties of the foam barrier/interceptor structure.

As noted above, the pre-use foam composition expands to form the foam barrier/interceptor structure when contacted with a triggering agent. As used herein a triggering agent may be any agent that when contacted with the pre-use foam composition triggers the gas providing agent to release a gas, and a foam structure to form.

In certain instances, the triggering agent may activate the pre-use foam composition during use of the absorbent article. For example, in one embodiment, the triggering agent may be a body exudate such as urine, menses, sweat, vaginal secretions, feces, and combinations thereof. Upon contact with the body exudate, or more specifically, moisture present in the body exudate, the gas providing agent is triggered, and a non-toxic gas is released. Other examples of in-use triggering agents include heat (e.g., body heat from the user of the absorbent article) and pressure (e.g., pressure exerted on the absorbent article when the body of the article user contacts the article). Combinations of triggering agents may also be required to activate the pre-use foam composition.

Alternatively or in addition, the triggering agent may activate the gas providing agent prior to contact with a body exudate, such as through an action of the absorbent article user. For instance, the user may trigger the gas providing agent by applying pressure to a portion of the article such as by pushing or squeezing the article prior to use. The user may also activate the gas providing agent by, for example, removing a release strip on the absorbent article that releases a compound or combination of compounds that react to produce a gas and a foam barrier/interceptor structure.

As noted above, the foam barrier/interceptor structures of the present disclosure are typically formed on the body-facing side of the absorbent article (e.g., on the topsheet). By locating the foam structure on the body-facing side of the absorbent article, the foam structure is effective at blocking the flow of bodily exudates that are not initially absorbed by the absorbent article across the article's topsheet, thus preventing leakage and soiling of the clothing of the article's wearer. Additionally, by locating the foam structure on the body-facing side of the article, the foam structure may also act as an interceptor, absorbing or intercepting bodily exudates that may be located on the wearer's body. This improves both the comfort level of the article's wearer, as well as further reduces the possibility of leakage.

The foam structures of the present disclosure are advantageously not only effective at preventing leakage, but also have improved fit against the wearer's body, and thus are also comfortable to the wearer. Additionally, the pre-use foam composition may optionally comprise a skin feel enhancing agent, which may also act to improve the skin feel of not only the pre-use foam composition, but also the foam structure.

The foam structures of the present disclosure also are advantageously very stable. As used herein, the term “stable foam structure” is intended to include foam barrier/interceptor structures that are capable of maintaining their barrier and/or interceptor properties during wear. Generally, to maintain these properties, the shape of the barrier and/or interceptor will continually deform due to deformational forces seen during wear, but will not permanently collapse. Preferably, the barrier and/or interceptor will not permanently collapse under usage pressures of up to about 6 psi. Typically, the stable foam structures have a yield stress of about 30,000 dynes/cm² or less and a viscosity of at least about 75,000 mPa*s.

The stability of the foam structures may be achieved either through use of a foam forming agent that also has foam stabilizing properties, or through incorporation of a foam stabilizing agent into the pre-use foam composition. The resulting foam structure is better able to maintain its gap filling foam volume, thus increasing the longevity of the foam, as compared to many commercially available foams. Furthermore, the foam generating agent and/or the foam stabilizing agent enables the foam structure to better withstand collapse due to pressures exerted on the foam, such as deformational forces caused by movement of the wearer.

Preferably, the stable foam produced upon contact of the pre-use foam composition with a triggering agent is a wet liquid foam. The foams described herein may comprise up to about 95% by weight water, which may come from water present in the triggering agent that is incorporated into the foam barrier or interceptor structure and/or from liquid included in the pre-use foam composition. Advantageously, the foam will have physical properties similar to those of commercial foaming shaving gels and creams. Preferably, the viscosity of the foam is sufficient to give the foam enough stability that the foam structure adequately fills the gaps between the wearer and the absorbent article. The foam will preferably have a viscosity at least about 75,000 mPa*s, and more preferably will have a viscosity at least about 85,000 mPa*s.

Additionally, the foam will preferably have a yield stress low enough so that normal deformational forces seen by the article during wear do not result in unpleasant pressure points on the wearer leading to the article being perceived as uncomfortable by the wearer. Typically, the yield stress of the foam is greater than 0 dyne/cm² to about 30,000 dyne/cm² or less, and more preferably to about 10,000 dyne/cm² or less.

The foam structure preferably remains for the lifetime of the product, and may be the result of either the one-time formation of a stable barrier/interceptor structure, or alternately, may be the result of the continuous formation of a foam barrier/interceptor structure. For example, the foam may be produced over an extended period of time such that if the bubbles of the foam are burst when the wearer sits or otherwise exerts pressure on the absorbent article, they will reform upon the release of the pressure to again form a foam structure. The production of the foam barrier or interceptor structure will continue until one of the gas providing agent or foam forming agent is consumed.

### Pre-Use Foam Composition

As noted above, the absorbent articles described herein comprise a pre-use foam composition comprising components that act as a gas providing agent, components that act as a foam forming agent, and components that act as a foam stabilizing agent. As used herein, the term “composition” is intended to encompass products resulting from the mixing or combining of various components or ingredients. When contacted with a triggering agent, the components of the pre-use foam composition interact to produce the foam barrier and/or interceptor structure. As noted above, in addition to the gas providing and foam forming agents, the pre-use foam composition of the present disclosure advantageously may further include foam forming adjuvants such as foam stabilizing agents and skin feel agents that improve the feel of the pre-use foam composition and/or foam barrier/interceptor structure against the skin of the article's wearer. Other examples of foam forming adjuvants that may be included in the pre-use foam composition include, for example, binding agents, trigger control agents, surface enhancing agents, sensual enhancing agents, and the like. In one preferred embodiment, the composition comprises a gas generating agent, a foam forming agent, a foam stabilizing agent, optionally a skin feel enhancing agent and a trigger control agent, and combinations thereof.

In certain embodiments, the pre-use foam composition may comprise components that have multiple functions. For instance, the composition may comprise a single component that acts as two or more of a gas providing agent, a foam forming agent, and a foam stabilizing agent, and/or other components, such as a skin feel enhancing agent, a trigger control agent, and the like. In one embodiment, the pre-use foam composition may comprise a single ingredient that acts as a gas providing agent, a foam forming agent, and a foam stabilizing agent. More typically, however, the composition comprises more than one component, with at least one component preferably (but not always) having multiple functions. Examples of suitable pre-use foam composition components that may have multiple functions are set forth hereinafter.

The components of the pre-use foam composition are advantageously incorporated into the absorbent articles in such a way that the foam barrier and/or interceptor structure is not produced until contact with a triggering agent. The composition components are thus preferably incorporated into the absorbent article in an effective form and mixed in an effective way to so that the foam barrier/interceptor structure is formed in the desired location and has the desired properties.

For instance, certain composition components may react or interact with other composition components prior to contact with the triggering agent unless they are physically separated from the other components or in a form such that no reaction with other composition components will occur. One example of this is acidic gas providing agents and basic gas providing agents. As discussed hereinafter, acidic and basic gas providing agents desirably react upon contact with the triggering agent to produce a gas which combines with the foam forming and foam stabilizing agents to produce the barrier/interceptor structure. However, if one or both of the acidic or basic gas generating agents is in a form such that it is capable of reacting with the other agent to produce a gas (e.g., in liquid form), the gas providing agents present in the pre-use foam composition may be depleted prior to contact with the triggering agent, rendering the pre-use foam composition ineffective.

It is thus advantageous to consider both form and physical properties when selecting components for inclusion in the pre-use foam composition. For example, the components of the pre-use foam composition may be in a solid (e.g., dry), semi-solid, and/or liquid form, and may be incorporated into or onto the absorbent article neat, in encapsulated form, or in combinations thereof. Typically, liquid composition components are encapsulated. Encapsulating liquid components helps isolate the liquid component from other composition components, preventing premature reaction with other composition components and migration of the liquid component into the absorbent core of the article. Other examples of suitable forms of the pre-use foam composition components are described hereinafter.

The shell material used for encapsulation may be suitably constructed of a material such that it will release the encapsulated material (i.e., the gas providing agent, foam forming agent, foam stabilizing agent, and/or other composition components) upon contact with the triggering agent. In certain embodiments, triggering agents containing liquids, such as urine or other body exudates, may cause the shell material to solubilize, disperse, swell, or disintegrate, or the shell material may be liquid or exudate permeable such that it disintegrates or discharges the encapsulated material upon contact with the exudate. In other embodiments, the shell material may be sensitive to heat or pressure, such that the shell material disintegrates or discharges the encapsulated material upon application of heat or pressure.

Examples of shell materials suitable for encapsulating the pre-use foam composition components include those described hereinbelow for formation of gas-filled microsphere shells, as well as cellulose-based polymeric materials (e.g., ethyl cellulose), carbohydrate-based materials (e.g., starches and sugars) and materials derived therefrom (e.g., dextrins and cyclodextrins). Other examples of encapsulating materials include water soluble synthetic or natural polymers such as polyacrylates (e.g. encapsulating polyacrylic acid), cellulosic gums, polyurethane and polyoxyalkylene polymers.

Other specific examples of biodegradable and physiologically compatible shell materials include proteins, such as albumin, gelatin, fibrinogen, collagen, as well as their derivatives, such as succinylated gelatin, crosslinked polypeptides, reaction products of proteins with polyethylene glycol (e.g., albumin conjugated with polyethylene glycol), chitin, chitosan, pectin, biodegradable synthetic polymers such as polylactic acid, copolymers consisting of lactic acid and glycolic acid, polycyanoacrylates, polyesters, polyamides, polylcarbonates, polyphosphazenes, polyamino acids, poly-ξ-caprolactone as well as copolymers consisting of lactic acid and ξ-caprolactone and their mixtures, are suitable. Especially suitable are albumin, polylactic acid, copolymers consisting of lactic acid and glycolic acid, polycyanoacrylates, polyesters, ppolycarbonates, polyamino acids, poly-ξ-caprolactone as well as copolymers consisting of lactic acid and ξ-caprolactone.

The shell thickness may vary depending upon the material encapsulated, and is generally manufactured to allow the encapsulated component to be covered by a thin layer of encapsulation material, which may be a monolayer or thicker laminate, or may be a composite layer. The layer should be thick enough to resist cracking or breaking of the shell during handling or shipping of the product or during wear which would result in breakage of the encapsulating material. The material should also be constructed such that humidity from atmospheric conditions during storage, shipment, or wear will not cause a breakdown of the microencapsulation layer.

Additionally, it is preferable that the pre-use foam composition be relatively stable. For example, the pre-use foam composition preferably has properties such that it is relatively immobile and localized on the body-facing surface of the absorbent article. Stability of the pre-use foam composition is desirable to avoid migration of the composition into the interior of the absorbent product. Additionally, by providing a composition that is relatively immobile, the location where the foam barrier/interceptor structure is formed can be controlled.

The immobility of the pre-use foam composition may be achieved through use of adhesives, such as described below, to adhere the composition to the desired portion of the topsheet. Alternately or in addition, the physical properties of the pre-use foam composition components may be such that migration of the composition to undesired locations of the absorbent article is minimized. Preferably, the pre-use foam compositions are solid, or more often semi-solid, at 20° C., i.e., at ambient temperatures. By “semisolid” is meant that the composition has a rheology typical of pseudoplastic or plastic liquids. When no shear is applied, the compositions can have the appearance of a semi-solid but can be made to flow as the shear rate is increased. This is due to the fact that, while the composition typically contains primarily solid components, it may also includes some minor liquid components. Preferably, the pre-use foam compositions have a zero shear viscosity between about 1.0×10⁶ centipoise and about 1.0×10⁸ centipoise. More preferably, the zero shear viscosity is between about 5.0×10⁶ centipoise and about 5.0×10⁷ centipose. As used herein, the term “zero shear viscosity” refers to a viscosity measured at very low shear rates (e.g., 1.0 sec⁻¹) using plate and cone viscometer (a suitable instrument is available from TA Instruments of New Castle, Del. as model number CSL 100).

Preferably, the pre-use foam composition is at least semi-solid at room temperature to minimize composition migration. In addition, the compositions preferably have a final melting point (100% liquid) above potential “stressful” storage conditions that can be greater than 45° C. By being solid or semisolid at ambient temperatures, the compositions do not have a tendency to flow and migrate to a significant degree to undesired locations of the article to which they are applied. As a result, the foam structure is formed in the desired location on the article.

To enhance immobility of the pre-use foam compositions, the viscosity of the composition is preferably as high as necessary to prevent flow within the article to undesired locations. Suitable viscosities for the pre-use foam compositions will typically be from about 5 to about 500 centipoise, preferably from about 5 to about 300 centipoise, more preferably from about 5 to about 100 centipoise, measured at 60° C. using a rotational viscometer (a suitable viscometer is available from Lab Line Instruments, Inc. of Melrose Park, Ill. as Model 4537). The viscometer is operated at 60 rpm using a number 2 spindle.

Preferably, the pre-use foam composition comprises from about 0.1% by weight to about 99% by weight of a gas providing agent, from about 1.0% by weight to about 99% by weight of a foam forming agent, from about 0.01% by weight to about 99% by weight of a foam stabilizing agent. Optionally, the composition may further comprise from about 0.001% by weight to about 99% by weight of other components, such as skin feel enhancing agents, binding agents, trigger control agents, surface enhancing agents, sensual enhancing agents, and the like. In one particular embodiment, the pre-use foam composition comprises about 30% by weight of a gas providing agent, about 30% by weight of a foam forming agent, about 30% by weight of a foam stabilizing agent, and about 10% by weight of other components.

Unless otherwise indicated, the amounts of pre-use foam composition components incorporated into the absorbent articles described herein are given in percent by weight of the pre-use foam composition, i.e., by total weight of the pre-use foam composition prior to contact with a triggering agent and formation of a foam barrier or interceptor structure.

### Gas Providing Agents

As noted above, the pre-use foam composition comprises a gas providing agent. As used herein, the term “gas providing agent” refers to any material or compound that is capable of releasing or producing gas upon contact with a triggering agent. For instance, the gas providing agent may be a solid material (e.g., a crystal matrix) in which a gas is trapped, such that gas is released upon contact with a triggering agent. Alternatively, the gas providing agent may be a mixture of compounds that when contacted or mixed together and contacted with a triggering agent, react and produce a non-toxic, gaseous reaction product. In each instance, the gas providing agents used in accordance with the present disclosure are substantially non-hazardous when contacted with human skin.

For purposes of the present disclosure, it should be understood that the form in which the gas providing agent is present in the absorbent articles may vary depending on the desired triggering agent.

As noted above, the gas providing agent may be a mixture of compounds which when contacted or mixed together and contacted with a triggering agent, react and produce a non-toxic, gaseous reaction product. For instance, in one embodiment, the gas providing agent may comprise at least one acid and at least one base (or alkaline material), which react together to produce a gas. Preferably, the gas is an inert, non-toxic gas, such as carbon dioxide, nitrogen, or oxygen gas, among others. The exact gas produced by the gas providing agent is not critical, so long as the gas produced is substantially non-harmful to the skin of the wearer in the quantities generated.

In certain instances, the gas providing agent may produce a gas through an effervescent reaction. Effervescent reactions are well known, and may include the reaction of carbonate salts such as sodium carbonate and/or sodium bicarbonate with acidic materials such as citric, malic, or fumaric acid, or the like, so that a gas is generated by the resulting neutralization reaction.

Examples of suitable acids that may be reacted to produce a gas include C₂-C₂₀ organic mono- and poly-carboxylic acids and especially alpha- and beta-hydroxycarboxylic acids; C₂-C₂₀ organophosphorus acids such as phytic acid; C₂-C₂₀ organosulfur acids such as toluene sulfonic acid; and peroxides such as hydrogen peroxide. Typical hydroxycarboxylic acids include adipic, glutaric, succinic, tartaric, malic, maleic, lactic, salicylic, and citric acids, as well as acid forming lactones such as gluconolactone and gluccrolactone. Other examples of suitable acids include formic acid, acetic acid, propanoic acid, butyric acid, valeric acid, oxalic acid, malonic acid, glycolic acid, aspartic acid, pimelic acid, fumaric acid, phthalic acid, isophthalic acid, terphthalic acid, glutamic acid, hydroxy acrylic acid, alpha hydroxy butyric acid, glyceric acid, tartronic acid, hydroxy benzoic acid, gallic acid, mandelic acid, tropic acid, ascorbic acid, gluconic acid, cinnamic acid, benzoic acid, phenylacetic acid, nicotinic acid, kainic acid, sorbic acid, pyrrolidone carboxylic acid, trimellitic acid, benzene sulfonic acid, potassium dihydrogen phosphate, sodium sulfite, sodium dihydrogen phosphate, potassium sulfite, sodium pyrosulfite, acidic sodium hexametaphosphate, acidic potassium hexametaphosphate, acidic sodium pyrophosphate, acidic potassium pyrophosphate, sulfamic acid, and phosphoric acid, among others. Preferably, the acid is one that has high fluid solubility and is non-toxic. The acids can be used alone or in combination with each other.

Suitable base materials include salts of carbonates and bicarbonates, alkaline peroxides (e.g. sodium perborate and sodium percarbonate) and azides (e.g. sodium azide). Other suitable alkaline carbonate salts are salts such as sodium bicarbonate, sodium carbonate, sodium sesquicarbonate, potassium bicarbonate, potassium carbonate, potassium sesquicarbonate, magnesium carbonate, ammonium bicarbonate, ammonium carbonate, ammonium sesquicarbonate, and calcium carbonate, among others. Preferably, the base material is one that has high fluid solubility and is non-toxic. The base materials can be used alone or in combination with each other.

The gas providing agent may comprise any acid/base combination capable of reacting upon contact with a triggering agent to produce a non-toxic gas. One specific example of a suitable acid/base combination is shown in equation (1):

NaHCO₃+KHC₄H₄O₆ [symbol] KNaC₄H₄O₆+H₂O+CO₂ (1)

In equation (1), sodium bicarbonate and potassium bitartrate react in the presence of a liquid (e.g., urine) to form carbon dioxide gas and by-products. The production of carbon dioxide, in combination with a foam forming agent, allows the formation of a foam barrier or interceptor structure.

Another specific example of a suitable acid/base combination for use in absorbent articles described herein is shown in equation (2):

NaAl(SO₄)₂+3NaHCO₃ [symbol] Al(OH)₃+2Na₂SO₄+3CO₂ (2)

In equation (2), sodium aluminum sulfate and sodium bicarbonate react in the presence of liquid (e.g., urine) to form carbon dioxide gas and by-products. Other acids, such as those listed above, can be used in combination with sodium bicarbonate to produce a gas in accordance with the present disclosure. Other preferred acid/base combinations include, for example, citric acid and sodium bicarbonate.

In order to fully protect against any damage to the skin by the acid/base reaction, it is desirable that the resulting acid/base mixture in the presence of the triggering agent should have a pH of from about 4.5 to about 7.5. A buffering agent could optionally be utilized in combination with the acid/base combination to help control the resulting pH of the acid/base mixture.

The amount of acid and base incorporated into the absorbent article will vary depending on the desired amount of gas to be generated. The needed amount of gas will vary depending on the physical properties of the foam barrier structure to be produced, and the volume of foam desired. Typically, the volume of foam produced should be sufficient to form an effective foam structure during use. Typically, the amount of foam produced is from about 1 mL to about 30 mL. The amounts of the acid and base which can be mixed to produce the desired volume of carbon dioxide (or other gas) when dissolved by water in the triggering agent can be determined by stoichiometry. Typically, however, the pre-use foam composition comprises at least about 0.1 g of acid, and more typically from about 0.1 g to about 15 g of acid, and at least about 0.1 g of base, and more typically from about 0.1 g to about 15 g of base.

As noted above, the acids and bases used in the articles described herein should preferably be substantially non-reactive with each other until contacted with the triggering agent. Therefore, depending on the type of triggering agent used, the gas providing agent could be in the form of a mixture of acid powders and base powders, liquid acids and liquid bases, or combinations thereof. The acids and bases can be introduced into the absorbent article neat and/or in microencapsulated form. Typically, if a liquid acid or base is utilized, it will be in microencapsulated form.

In a particular embodiment, the acid and base are separately encapsulated into microencapsulated shells. A combination of encapsulated shells containing acid and encapsulated shells containing base are then introduced into or onto the absorbent article, as described herein. It should be recognized that with some acid/base combinations, it may be possible to first mix the acid and base together and then introduce the mixture into an encapsulated shell which can be introduced into an absorbent article in accordance with the present disclosure. In a separate embodiment, the acid and base are dispersed neat into or onto the absorbent article of the present disclosure. It will also be recognized that a combination of encapsulated and neat acids and bases could be utilized. Any combination of compounds, such as an acid and a base, that are substantially non-reactive with each other until contacted with the triggering agent and that are capable of reacting to form at least one non-toxic gaseous product upon wetting (or otherwise triggered) may be used as a gas providing agent.

In one preferred embodiment, the gas providing agent comprises a mixture of acid powders and base powders in neat and/or encapsulated form. Upon wetting by a triggering agent, the acid and base powders react with one another to produce a gaseous product. In this embodiment, the liquid used to trigger the reaction may come from fluids present in a triggering agent, such as urine, menses, vaginal secretions, fecal matter, sweat, and the like.

In an alternate embodiment, the gas providing agent may comprise a liquid acid and/or a liquid base. As noted above, if a liquid acid and/or liquid base is utilized, it is generally preferable for the liquid gas providing agent to be in microencapsulated form. This prevents the liquid gas providing agent from being absorbed into the absorbent article and from prematurely reacting to produce the gas.

In embodiments where the gas providing agent comprises a microencapsulated acid and/or base, the acid and/or base may be released from the encapsulated shell by contact with the triggering agent. For instance, liquid from a triggering agent may act to dissolve the microencapsulated shell, thus releasing the acid and/or base gas providing agents, which subsequently react to produce the gas. Alternately, the triggering agent may be in the form of pressure and/or heat, such as discussed above, which when applied to the encapsulated shell containing the gas providing agent, ruptures the shell and releases the acid and/or base gas providing agents, which subsequently react to produce the gas.

In another embodiment, the gas providing agent comprises a liquid-soluble solid material produced in such a manner that a pressurized gas is trapped within cells located in the solid material. When the solid material having pressurized gas-containing cells is contacted with a triggering agent, such as urine or other liquid-containing triggering agent, it begins to dissolve and the pressurized gas is released from the cells during dissolution of the solid material. This gas can interact with the foam forming agent to produce a foam barrier or interceptor structure as described herein.

In this embodiment, the liquid-soluble solid material may comprise a sugar compound such as a mono-saccharide, di-saccharide, or poly-saccharide that has been infused with a gas that is substantially non-reactive with human skin. Suitable gases for infusion into a solid material include, for example, carbon dioxide, air, nitrogen, noble gases such as argon, helium, neon, krypton, xenon, and radon, halogenated hydrocarbons, other substantially inert gases, and combinations thereof. Specific examples of saccharides that can be used in accordance with the present disclosure include glucose, fructose, sucrose, lactose, maltose, dextrin, cyclodextrin, and the like, alone or in combination. Also, a mixture of sucrose with corn syrup (containing glucose, maltose, and dextrin) can be used in accordance with this embodiment of the present disclosure to produce a gas-containing gas providing agent. Other examples of compounds that are capable of being prepared in such a manner as to trap pressurized gas in cells include, for example, water soluble compounds such as salts, alkali halides, and alkaline earth metal halides. Specific salts useful in the present disclosure include, for example, sodium chloride, potassium chloride, potassium bromide, lithium chloride, cesium chloride, and the like. Typically, the cells containing the pressurized gas have a diameter of from about 5 micrometers to about 100 micrometers.

The substantially non-reactive gas can be infused into the cells of the liquid-soluble solid material to produce a gas providing agent useful in the present disclosure by first heating the starting material, such as a sugar, in a small amount of water until the material is dissolved. After dissolution of the material, the water is evaporated off leaving the material in a molten state. The molten material is then gasified by introducing a suitable gas, such as carbon dioxide, at a superatmospheric pressure into a sealed vessel containing the molten material. The molten material is agitated during gasification to ensure intimate contact between the molten material and the gas. Pressures of, for example, between about 50 psig (340 kPa) and about 1000 psig (6890 kPa) may be utilized to infuse the gas into the molten material. After gas infusion, the molten material is allowed to solidify while maintained in the sealed vessel to produce a gas providing agent. A suitable procedure of producing a gas containing solid material is fully set forth in U.S. Pat. No. 4,289,794, which is hereby incorporated by reference. The above procedure can produce solid gas providing agents containing cells of pressurized gas from about 50 psig (340 kPa) to about 900 psig (6200 kPa) which, when exposed to liquid such as urine, allow the release of the trapped gas. This trapped gas, when released, can interact with the foam forming agent as described herein.

In another embodiment, the gas providing agent comprises a volatile compound entrapped within a liquid-soluble solid material. By entrapping the volatile compound in a liquid-soluble solid material, the volatility of the compound is controlled until the liquid-soluble solid material is contacted with a triggering agent, such as urine or other liquid-containing triggering agents, and begins to dissolve, releasing the volatile compound. Released volatile gas can interact with the foam forming agent and foam stabilizing agent to produce a foam barrier or interceptor structure. Alternately, if the volatile compound is a volatile liquid, once released from the solid material, the volatile liquid may evaporate, resulting in foaming of the foam forming material. Examples of volatile fluid-forming agents include, for example, aliphatic hydrocarbons such as ethane, ethylene, propane, propene, butane, isobutene, neopentane, acetylene, hexane, heptane, and mixtures thereof. The encapsulation of volatile liquids is described in U.S. Pat. No. 3,615,972, herein incorporated by reference.

Any volatile compound may be entrapped, so long as it is it is non-toxic and relatively non-harmful to human skin. Preferably, the volatile compound is either odor free, or has a pleasant odor. The volatile compound may be a gas at room and/or body temperature, or alternatively may be a liquid at room and/or body temperature. Examples of suitable volatile compounds include, for example, hydrocarbons having 4 to 6 carbon atoms, such as isopentane, pentane, hexane, and butane, as well as perfumes, essential oils, volatile organic compounds (VOCs), and combinations thereof. Examples of suitable VOCs include those having a vapor pressure greater than 0.02 Torr at around body temperature. In one embodiment, the VOCs have a vapor pressure of about 760 torr at around body temperature.

The volatile compound may be entrapped in a liquid soluble solid material, such as described above. Methods for entrapping volatile compounds in a sugar or salt matrix are also described in U.S. Pat. No. 3,970,766, herein incorporated by reference.

In particular embodiments, the solid material containing the entrapped gas or volatile compound may be introduced into or onto the absorbent article in neat form. Alternately, the solid material may itself be encapsulated, and the encapsulated solid material introduced into or onto the absorbent article. Combinations of encapsulated and unencapsulated (i.e., neat) solid material may also be used.

The amount of solid material containing an entrapped gas or volatile compound that is incorporated into the absorbent article will vary depending on the desired amount of gas to be released. As noted above, the needed amount of gas will vary depending on the physical properties of the foam barrier or interceptor structure to be produced, and the volume of foam desired. Typically, from about 0.1 grams to about 15 grams, and more preferably from about 1 gram to about 5 grams of solid material containing an entrapped pressurized gas or volatile compound is incorporated into the absorbent articles described herein.

In certain embodiments, the gas providing agent may comprise a gas-filled microsphere. In these embodiments, a gas is released from the microsphere upon contact with the triggering agent, and interacts with the foam forming agent to form a foam barrier structure. Methods for producing gas-filled microspheres are known in the art and described in, for example, WO 96/04018, WO 96/40279, U.S. Pat. Nos. 5,552,133, 6,068,857, and 5,674,469, herein incorporated by reference in their entirety. Suitable gases for infusion into a the microsphere include, for example, carbon dioxide, air, nitrogen, noble gases such as argon, helium, neon, krypton, xenon, and radon, halogenated hydrocarbons, other substantially inert gases, and combinations thereof.

Materials that are be suitable for forming the microsphere shells include, but are not limited to, lipids, proteins (both naturally occurring and synthetic amino acid polymers), synthetic organic polymers, and mixtures or copolymers thereof. Lipid shells can be formed from either naturally occurring or synthetic lipids, for example, phospholipids, such as phosphoglycerides, phosphatidic acid, phosphatidylcholine, phosphatidyl serine, phosphatidylethanolamine, phsophatidyl inositol, phosphatidylglycerol, diphosphatidyl-glycerol (cardiolipin); glycolipids, such as cerebrosides, galactocerebrosides, gluco-cerebrosides, sphingomyelin, sphingolipids, derivatized with mono-, di-, and trihexosides, sulfatides, glycosphingolipid, and lysophosphatidylcholine; unsaturated fatty acids, such as palmitoleic acid, oleic acid, vaccenic acid, linoleic acid, α-linolenic acid, and arachidonic acid; saturated fatty acids, such as myristic acid, palmitic acid, staric acid, arachidic acid, behenic acid, lignoceric acid, and cerotic acid; mono-, di-, and triglycerides; and steroids, such as cholesterol, cholesterol esters, cholestanol, ergosterol, coprostanol, squalene, and lanosterol.

In one embodiment, the gas providing agent may comprise other compounds, such as blowing agents that decompose into gaseous products upon the application of moderate amounts of heat. For example, the gas providing agent may comprise ammonium carbamoylsulfonate (NH₄O₃SCONH₂), which decomposes into gaseous products upon heating to temperatures in the range of from about 40° C. to about 120° C. Other examples of suitable gas providing agents include compounds that decompose at around body temperature, such as ammonium carbamate and carbonate, which decompose into gaseous products upon heating to temperatures in the range of from about 30° C. to about 40° C. These gas providing agents may be incorporated into or onto the absorbent article in neat form and/or may be encapsulated prior to incorporation into or onto the absorbent article.

As noted above, the gas providing agent may be incorporated into or onto the absorbent article neat, in encapsulated form, or in combinations thereof. The shell material used for encapsulation may be suitably constructed of a material such that it will release the encapsulated material (i.e., the acid, base, entrapped gas, entrapped volatile compound, or other gas providing agent, and/or foam forming agent) upon contact with the triggering agent. In certain embodiments, triggering agents containing liquids, such as urine or other body exudates, may cause the shell material to solubilize, disperse, swell, or disintegrate, or the shell material may be liquid or urine permeable such that it disintegrates or discharges the encapsulated material upon contact with the liquid. In other embodiments, the shell material may be sensitive to heat or pressure, such that the shell material disintegrates or discharges the encapsulated material upon application of heat or pressure. Examples of suitable shell materials are described hereinbefore.

### Foam Forming Agents

The pre-use foam composition also comprises at least one foam forming agent. When a gas, such as carbon dioxide, is released by the gas providing agent upon contact with the triggering agent, the gas expands the liquid present that includes the foam forming agent and a foam is produced. As the foam expands and swells, it fills the void between the absorbent article and the body of the user, creating an interceptor and/or a barrier to leakage. Alternately, the foam need not expand to fill the entire void, but rather, may expand sufficiently to form a raised barrier structure on the body facing surface of the topsheet.

As noted above, in some embodiments, the foam forming agent may have a dual function. For instance, in one preferred embodiment, the foam forming agent may also act as a foam stabilizing agent, stabilizing the foam produced when the pre-use foam composition is contacted by the triggering agent. In these embodiments, it is not necessary for the pre-use foam composition to further comprise an additional foam stabilizing agent, as the foam forming agent will also act to stabilize the foam barrier/interceptor structure.

A variety of compounds may act as foam forming agents. For instance, the foam forming agent may be a surfactant that facilitates the generation of a foam. Examples of suitable surfactants include both ionic surfactants and non-ionic surfactants.

The amount of foam forming agent incorporated into the absorbent articles described herein will vary depending on the desired physical properties of the foam, the volume of foam to be produced, and the other components of the pre-use foam composition, but should be enough to produce the desired foam structure. Typically, the amount of foam forming agent in the pre-use foam composition is from about 1% by weight to about 99% by weight, more typically from about 30% by weight to about 90% by weight, and still more typically about 30% by weight of the pre-use foam composition.

The foam forming agent may be incorporated into or onto the absorbent article neat and/or may be encapsulated, and the encapsulated foam forming agent incorporated into or onto the absorbent article. The foam forming agent may optionally comprise a combination of neat and encapsulated components. Optionally, the foam forming agent, gas providing agent, and/or foam stabilizing agent may be combined and encapsulated together. If the foam forming, foam stabilizing, and gas providing agents are encapsulated together, it is preferable that the foam forming agent, foam stabilizing agent, and gas providing agent do not foam until contact with a triggering agent.

### Viscoelastic Surfactants

In one preferred embodiment, the foam forming agent may comprise a viscoelastic surfactant. As used herein, the term “viscoelastic surfactant” refers to a surfactant or combination of surfactants that is capable of forming a viscoelastic fluid in solution. Advantageously, viscoelastic surfactants may act as both a foam forming agent and a foam stabilizing agent. More particularly, viscoelastic surfactant systems have both viscous and elastic properties. Without wishing to be bound to any particular theory, it is believed that elastic nature of viscoelastic surfactants may help stabilize the foam barrier/interceptor structure produced when the pre-use foam composition is contacted with a triggering agent, as described herein.

Examples of suitable viscoelastic surfactants are known in the art and described in, for example, U.S. Pat. Nos. 5,258,137 and 5,965,502, as well as U.S. Patent App. No. 2004/0102569, all herein incorporated by reference.

In one embodiment, the viscoelastic surfactants can be either ionic or nonionic. In general, an ionic viscoelastic surfactant comprises a surfactant compound having a hydrophobic moiety chemically bonded to an ionic, hydrophilic moiety (hereinafter referred to as a “surfactant ion”) and an amount and type of a counterion having a moiety capable of associating with the surfactant ion sufficient to form a viscoelastic surfactant. A nonionic viscoelastic surfactant comprises a surfactant ion having a hydrophobic moiety chemically bonded to a nonionic, hydrophilic moiety.

Examples of ionic surfactant compounds are represented by the formula:

R₁(Y⁺)X⁻ or R₁(Z⁻)A⁺

wherein R₁(Y⁺) and R₁(Z⁻) represent surfactant ions having a hydrophobic moiety represented by R₁ and an ionic, solubilizing moiety represented by the cationic moiety (Y⁺) or the anionic moiety (Z⁻) chemically bonded thereto. X⁻ and A⁺ are the counterions associated with the surfactant ions.

In general, the hydrophobic moiety (i.e., R₁) of the surfactant ion is hydrocarbyl or inertly substituted hydrocarbyl wherein the term “inertly substituted” refers to hydrocarbyl radicals having one or more substituent groups, e.g., halo groups such as —F, —Cl, or —Br or chain linkages, such as a silicon linkage (—Si—), which are inert to the aqueous liquid and components contained therein. Typically, the hydrocarbyl radical is an aralkyl group or a long chain alkyl or inertly substituted alkyl, which alkyl group is generally linear and have at least about 12, advantageously at least about 16, carbon atoms. Representative long chain alkyl and alkenyl groups include dodecyl (lauryl), tetradecyl (myristyl), hexadecyl (cetyl), octadecenyl (oleyl), octadecyl (stearyl) and the derivatives of tallow, coco and soya. Preferred alkyl and alkenyl groups are generally alkyl and alkenyl groups having from about 14 to about 24 carbon atoms, with octadecyl, hexadecyl, erucyl and tetradecyl being the most preferred.

The cationic, hydrophilic moieties (groups), i.e., (Y⁺), are generally onium ions wherein the term “ionium ions” refers to a cationic group which is essentiaily completely ionized in water over a wide range of pH, e.g., pH values from about 2 to about 12. Representative onium ions include quaternary ammonium groups, i.e., —N+O(R)₃; tertiary sulfonium groups, i.e., —S⁺(R)₂; quaternary phosphonium groups, i.e., —P⁺O(R)₃ and the like, wherein each R is individually a hydrocarbyl or inertly substituted hydrocarbyl. In addition, primary, secondary and tertiary amines, i.e., —NH₂, —NHR, or —N(R)₂, can also be employed as the ionic moiety if the pH of the aqueous liquid being used is such that the amine moieties will exist in ionic form or at least partially in ionic form. A pyridinium moiety can also be employed. Of such cationic groups, the surfactant ion of the viscoelastic surfactant is preferably prepared having quaternary ammonium, i.e., —N⁺(R)₃; a pyridinium moiety; an aryl- or alkaryl pyridinium; or imidazolinium moiety; or tertiary amine, —N(R)₂ groups wherein each R is independently an alkyl group or hydroxyalkyl group having from 1 to about 4 carbon atoms, with each R preferably being methyl, ethyl or hydroxyethyl.

Representative anionic, solubilizing moieties (groups) (Z⁻) include sulfate groups, i.e., —OSO₃—, ether sulfate groups, sulfonate groups, i.e.—SO₃—, carboxylate groups, phosphate groups, phosphonate groups, and phosphonite groups. Of such anionic groups, the surfactant ion of the viscoelastic surfactants is preferably prepared having a carboxylate or sulfate group. For purposes of this invention, such anionic solubilizing moieties are less preferred than cationic moieties.

Fluoroaliphatic species suitably employed include organic compounds represented by the formula:

R_{f}Z¹

wherein R_{f} is a saturated or unsaturated fluoroaliphatic moiety, preferably containing a F₃C⁻ moiety and Z¹ is an ionic moiety or potentially ionic moiety. The fluoroaliphatics can be perfluorocarbons. Suitable anionic and cationic moieties will be described hereinafter. The fluoroaliphatic moiety advantageously contains from about 3 to about 20 carbons wherein all can be fully fluorinated, preferably from about 3 to about 10 of such carbons. This fluoroaliphatic moiety can be linear, branched or cyclic, preferably linear, and can contain an occasional carbon-bonded hydrogen or halogen other than fluorine, and can contain an oxygen atom or a trivalent nitrogen atom bonded only to carbon atoms in the skeletal chain. More preferable are those linear perfluoroaliphatic moieties represented by the formula: CₙF₂ₙ₊₁ wherein n is in the range of about 3 to about 10. Most preferred are those linear perfluoroaliphatic moieties represented in the paragraphs below.

The fluoroaliphatic species can be a cationic perfluorocarbon and is preferably selected from the group consisting of CF₃(CF₂)ₙSO₂NH(CH₂)ₙN⁺R″₃X⁻; R_{F}CH₂CH₂SCH₂CH₂N⁺R″₃X⁻ and CF₃(CF₂)ᵣCONH(CH₂)ₛN⁺R″₃X⁻; wherein X⁻ is a counterion described hereinafter, R″ is lower alkyl containing between 1 and about 4 carbon atoms, r is about 2 to about 15, preferably about 2 to about 6, and s is about 2 to about 5.

The fluoroaliphatic species can be an anionic perfluorocarbon and is preferably selected from a member of the group consisting of CF₃(CF₂)ₚSO₂O⁻A⁺, CF₃(CF₂)ₚCOO⁻A⁺, CF₃(CF₂)ₚSO₂NH(CH₂)_{q}SO₂O⁻A⁺, and CF₃(CF₂)ₚSO₂NH(CH₂)_{q}COO⁻A⁺; wherein p is from about 2 to about 15, preferably about 2 to about 6, q is from about 2 to about 5, and A⁺ is a counterion described hereinafter.

The counterions (i.e., X⁻ or A⁺) associated with the surfactant ions are most suitably ionically charged, organic materials having ionic character opposite that of the surfactant ion, which combination of counterion and surfactant ion imparts viscoelastic properties to an aqueous liquid. The organic material having an anionic character serves as the counterion for a surfactant ion having a cationic, hydrophilic moiety, and the organic material having a cationic character serves as the counterion for the surfactant ion having an anionic, hydrophilic moiety. In general, the preferred counterions exhibiting an anionic character contain a carboxylate, sulfonate or phenoxide group wherein a “phenoxide group” is ArO⁻ and Ar represents an aromatic ring or inertly substituted aromatic ring. Representative of such anionic counterions which, when employed with a cationic surfactant ion, are capable of imparting viscoelastic properties to an aqueous liquid include various aromatic carboxylates such as o-hydroxybenzoate; m- or p-chlorobenzoate, methylene bis-salicylate and 3,4-, 3,5- or 2,4-dichlorobenzoate; aromatic sulfonates such as p-toluene sulfonate and naphthalene sulfonate; phenoxides, particularly substituted phenoxides; and the like, where such counterions are soluble; or 4-amino-3,5,6-trichloropicolinate. Alternatively, the cationic counterions can contain an onium ion, most preferably a quaternary ammonium group. Representative cationic counterions containing a quaternary ammonium group include benzyl trimethyl ammonium or alkyl trimethyl ammonium wherein the alkyl group is advantageously octyl, decyl, dodecyl, erucyl, and the like; and amines such as cyclohexyl amine. It is highly desirable to avoid stoichiometric amounts of surfactant and counterion when the alkyl group of the counterion is large. The use of a cation as the counterion is generally less preferred than the use of an anion as the counterion. Inorganic counterions, whether anionic or cationic, can also be employed.

The specific type and amount of surfactant ion and the counterion employed to prepare a viscoelastic surfactant are interrelated and are selected such that the combination imparts viscoelastic properties to an aqueous liquid. The combinations of surfactant ions and the counterions which will form a viscoelastic surfactant will vary and are easily determined by the methods described in U.S. Pat. No. 5,258,137.

Of the various surfactant ions and counterions which can be employed in preparing a viscoelastic surfactant, the preferred viscoelastic surfactants include those represented by the formula:

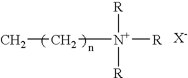

wherein n is an integer from about 13 to about 23, preferably an integer from about 15 to about 21; each R is independently hydrogen or an alkyl group, or alkylaryl, or a hydroxyalkyl group having from 1 to about 4 carbon atoms, preferably each R is independently methyl, hydroxyethyl, ethyl or benzyl, and X⁻ is o-hydroxy benzoate, m- or p-halobenzoate or an alkylphenate wherein the alkyl group is advantageously from 1 to about 4 carbon atoms. In addition, each R can form a pyridinium moiety. Especially preferred surfactant ions include cetyl trimethyl ammonium, oleyl trimethyl ammonium, erucyl trimethyl ammonium and cetyl pyridinium.

Other preferred viscoelastic surfactants include those represented by the formula:

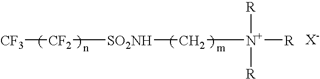

wherein n is an integer from about 3 to about 15, preferably from about 3 to about 8; m is an integer from about 2 to about 10, preferably from about 2 to about 5; R is as previously defined, most preferably methyl; and X⁻ is as previously defined.

Methods of preparing viscoelastic surfactants and of testing the viscoelastic properties of surfactants are described in U.S. Pat. No. 5,258,137.

Other examples of suitable viscoelastic surfactants include at least one oligomeric surfactant based on linked surfactant monomer subunits, each monomer subunit having the formula (R₁—X)ₚZₘ or R₁—Y; where X is a charged head group, Y is a zwitterionic polar headgroup (such as —N⁺(CH₃)₂—CH₃—COO⁻ or —N⁺(CH₃)₂—CH₃—OSO₃⁻), R₁ is a C₁₀-C₅₀ organic (preferably hydrocarbyl and/or aliphatic) tail group comprising a C₁₀-C₂₅ (preferably C₁₅-C₂₄) straight chain bonded at a terminal carbon atom thereof to respectively X or Y, Z is a counterion such as an alkali metal cation, and p and m are integers which ensure that the surfactant monomer is charge neutral.

The organic tail group may comprise only the straight chain. The straight chain may be a hydrocarbyl chain. In one embodiment the monomer straight chain is unsaturated. Preferably the oligomer is a dimer or a trimer.

X may be a carboxylate (—COO⁻), sulphate (—OSO₃⁻), sulphonate (—SO₃⁻), phosphate (—OPO₃²⁻), or a phosphonate (—PO₃²⁻) charged group. For the avoidance of doubt, it is hereby stated that when X is a carboxylate group the carbon atom of the carboxylate group is not counted with the carbon atoms of the organic group. The surfactant monomer may be a salt of oleic acid.

### Lathering Surfactants

The foam forming agent may further comprise at least one lathering surfactant. As used herein, “lathering surfactant” means a surfactant, which when combined with a liquid such as water and the gas released from the gas providing agent, generates a foam or lather. Preferably, the lathering surfactants used in the absorbent articles described herein are mild and non-irritating to skin. Typically, the amount of lathering surfactant in the pre-use foam composition is from about 1% by weight to about 99% by weight, more typically from about 5% by weight to about 40% by weight, and still more typically from about 10% by weight to about 25% by weight.

A wide variety of lathering surfactants are useful herein and include ionic lathering surfactants (e.g., cationic surfactants, anionic surfactants, and zwitterionic surfactants), and non-ionic lathering surfactants, and mixtures thereof. Especially preferred lathering surfactants are those that can be used as both a foam forming agent and a foam forming adjuvant, such as a foam stabilizing agent. Examples of such lathering surfactants are described in U.S. Publ. Pat. App. No. 2007/0072780, herein incorporated by reference.

A wide variety of anionic lathering surfactants are potentially useful herein. Nonlimiting examples of anionic lathering surfactants include those selected from the group consisting of alkyl and alkyl ether sulfates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, acyl glutamates, acyl sarcosinates, alkyl sulfoacetates, acylated peptides, alkyl ether carboxylates, acyl lactylates, anionic fluorosurfactants, and combinations thereof. Combinations of anionic surfactants can also be used effectively in the present disclosure. Examples of suitable anionic lathering surfactants are disclosed in, for example, U.S. Patent App. No. 2004/0147189, herein incorporated by reference.

One preferred example of anionic foam forming agents are acyl lactylates, which as discussed hereinafter, may also act as a foam stabilizing agent.

Cationic lathering surfactants are also useful in the articles of the present invention. Suitable cationic lathering surfactants include, but are not limited to, fatty amines, di-fatty quaternary amines, tri-fatty quaternary amines, imidazolinium quaternary amines, and combinations thereof. Suitable fatty amines include monalkyl quaternary amines such as cetyltrimethylammonium bromide. A suitable quaternary amine is dialklamidoethyl hydroxyethylmonium methosulfate. The fatty amines, however, are preferred. It is preferred that a lather booster is used when the cationic lathering surfactant is the primary lathering surfactant. Additionally, nonionic surfactants have been found to be particularly useful in combination with such cationic lathering surfactants.

Amphoteric lathering surfactants are also useful in the articles of the present invention. The term “amphoteric lathering surfactant,” as used herein, is also intended to encompass zwitterionic surfactants, which are well known to formulators skilled in the art as a subset of amphoteric surfactants.

A wide variety of amphoteric lathering surfactants can be used as foam forming agents. Particularly useful are those which are broadly described as derivatives of aliphatic secondary and tertiary amines, preferably wherein the nitrogen is in a cationic state, in which the aliphatic radicals can be straight or branched chain and wherein one of the radicals contains an ionizable water solubilizing group, e.g., carboxy, sulfonate, sulfate, phosphate, or phosphonate.

Nonlimiting examples of amphoteric or zwitterionic surfactants are those selected from the group consisting of betaines, sultaines, hydroxysultaines, alkyliminoacetates, iminodialkanoates, aminoalkanoates, and mixtures thereof. Examples of suitable amphoteric or zwitterionic surfactants are described in, for example, U.S Patent App. Publ. No. 2004/0147189, and U.S. Pat. No. 5,911,981.

Nonionic lathering surfactants useful herein include those selected from the group consisting of alkyl glucosides, alkyl polyglucosides, polyhydroxy fatty acid amides, alkoxylated fatty acid esters, sucrose esters, amine oxides, and mixtures thereof. Examples of suitable alkyl polyglucosides include octyl glucoside, decyl maltoside, and combinations thereof. Other examples of suitable nonionic surfactants include alkyl poly(ethylene oxide), copolymers of poly(ethylene oxide) and poly(propylene oxide) (commercially available as Poloxamers or poloxamines), cocamide MEA, cocamide DEA, cocamide TEA, and the like.

Other examples of suitable nonionic lathering surfactants are described in, for example, U.S Patent App. Publ. No. 2004/0147189, and U.S. Pat. No. 5,911,981, herein incorporated by reference.

In one particular example, the non-ionic lathering surfactant may be a fatty alcohol, which may also act as a foam stabilizing agent. Examples of suitable fatty alcohols include non-ethoxylated fatty alcohols, preferably those containing from 12 to 18 carbon atoms, and that are water-insoluble normally solid (i.e., at room temperature and atmospheric pressure) saturated or unsaturated alcohol. The preferred fatty alcohols include C₁₄-C₁₆ alcohols, most preferably myristyl and cetyl alcohol, as well as oleyl alcohol. The foam forming agent can optionally comprise two or more of the fatty alcohols. One preferred mixture is a blend of cetyl and myristyl alcohols. The ratio is not critical. Other examples of suitable fatty alcohols are described in, for example, U.S. Patent App. Publ. No. 2004/0018167, herein incorporated by reference.

### Foam Forming Adjuvants

As noted above, the pre-use foam composition may optionally comprise a foam forming adjuvant. As used herein, the term “foam forming adjuvant” is intended to include agents that improve the properties of the foam barrier/interceptor structure that is formed when the triggering agent contacts the pre-use foam composition. Suitable foam forming adjuvants include foam stabilizing agents, skin feel enhancing agents, surface enhancing agents, trigger control agents, hair and skin care agents, film forming agents, and the like.

### Foam Stabilizing Agents

As noted above, the pre-use foam composition may further comprise at least one foam stabilizing agent. The term “foam stabilizing agent” includes those agents that act to enhance the fluid bulk and surface and gas stability of the foam. Without wishing to be bound to any particular theory, it is believed that the foam stabilizing agent may increase the stability of the foam formed when the triggering agent contacts the pre-use foam composition by either strengthening the physical properties of the foam or by modifying unfavorable properties of the foam environment, or some combination thereof. For instance, the foam properties may be affected by adding a foam stabilizing agent that acts to slow the drainage of liquid or reduce water evaporation from the foam structure, increase the elasticity of the foam, decrease the diffusion of gas from the foam, increase the thickness of the layers of foam forming agents that make up the foam structure, increase the viscosity of the foam, or otherwise increasing the mechanical strength of the foam. Additionally, unfavorable foam environment properties may be modified by, for example, eliminating fomacidal soils and minimizing the depletion of the foam forming agent. Examples of suitable ways to increase foam stability are described in Lai and Dixit, “Additives for Foams” in Foam Theory, Measurements, and Applications, Science Series Vol. 57, pp. 315-338, herein incorporated by reference.

More particularly, it is believed the inclusion of a foam stabilizing agent helps maintain the foam barrier/interceptor structure as a wet, or spherical, foam. As noted above, the foam barrier/interceptor structure is created by dispersing the gas, released when the gas providing agent is contacted with the triggering agent, in the foam forming agent to form a foam structure. The mechanism of dispersing a gas in a surfactant-containing liquid is similar to the dispersion of two immiscible liquids during formation of an emulsion. Consequently, gas bubbles dispersed in a liquid are stabilized in the same manner as emulsions, e.g., by formation of surfactant layers at the gas-liquid interface. The surfactant layers keep the gas bubbles separated and prevent “coalescence,” i.e., the merging of small gas bubbles to form larger gas bubbles. In general, more dense and more compact surfactant layers form smaller bubbles and retard the coalescence mechanism.

It is well known that because of the very large density difference between the dispersed gas and the liquid, the gas bubbles rise to the top of the liquid. The enriched concentration of gas bubbles at the top of the liquid appear as “foam.” Initially, most if not all of the gas bubbles in the foam are spherical. There is sufficient space between each individual spherical gas bubble for the presence of the surfactant-containing liquid, and the foam behaves like an emulsion. This is termed a “wet foam.” Over time, however, the liquid present in the interstices between the individual gas bubbles drains out due to gravity. Depending on the nature and chemical structure of the surfactant in the liquid, lamellar liquid crystalline layers form and arrange at the gas-liquid interface. If the lamellar layers have a low viscosity, the surfactant-containing liquid between individual gas bubbles drains relatively easily, and the spherical form of the foam bubbles changes into a hexagonal form relatively quickly. Hexagonal bubbles quickly break. Foam in the hexagonal form is termed a “dry foam.” Dry foams are more unstable, which leads to a rapid reduction in foam volume due to rapidly breaking bubbles. However, if the lamellar surfactant layers have a high viscosity, the transition from a spherical foam to the hexagonal form is delayed. The inclusion of a foam stabilizing agent in the pre-use foam composition helps stabilize the foam barrier or interceptor structure over extended periods of time.

Thus, it is advantageous to incorporate into the pre-use foam composition at least one foam stabilizing agent. Preferably, the foam stabilizing agent is capable of maintaining the viscosity of the continuous phase of the foam at least about 75,000 mPa*s, and more preferably at least about 85,000 mPa*s (850 Poise).

As noted above, in some embodiments, the foam forming agent may also have foam stabilizing properties. In instances where the foam forming agent is also a foam stabilizing agent, inclusion of an additional foam forming agent in the pre-use foam composition is not required to achieve stability of the foam barrier/interceptor structure. However, in certain embodiments, it may be advantageous to include an additional foam stabilizing agent into the pre-use foam composition, even in instances where the foam forming agent also has foam stabilizing properties.

The amount and identity of the foam stabilizing agent incorporated into the absorbent articles is determined by the identity of the primary foaming agent (and the inherent foaming ability of the primary foaming agent), by the desired foam volume, and by the desired physical properties of the foam produced (e.g., the length of time the foam barrier or interceptor structure is to be maintained). Typically, however, the amount of a foam forming adjuvant that is used as a foam stabilizing agent present in the pre-use foam composition is from about 0.1% to about 50% by weight, more typically from about 0.1% to about 10% by weight, and more typically is about 0.1% to about 5% by weight.

Examples of suitable foam stabilizing agents include organic compounds, electrolytes, finely divided particles, polymers, bipolymers, liquid crystals, and combinations thereof. A discussion of these and other foam stabilizing agents as well as considerations for selection of suitable foam stabilizing agents can be found in Lai and Dixit, “Additives for Foams” in Foam Theory, Measurements, and Applications, Science Series Vol. 57, pp. 315-338.

In one embodiment, the foam stabilizing agent may be an organic compound such as, for example, a surfactant. As discussed above, certain surfactants may act as both foam forming agents and foam stabilizing agents. In particular, surfactants may act to stabilize foam by allowing for the formation of a closely packed foam which exhibits more elasticity and more resistance to drainage. Suitable organic additives include, for example, viscoelastic surfactants and fatty alcohols, such as those discussed hereinbefore, fatty acids, alkanol amides, amine oxides, betaines, sulfobetaines, phosphine oxides, alkyl sulfoxides, and combinations thereof.

Exemplary alkanol amides include, for example, lauric/myristic monoethanolamide and lauric/myristic diethanolamide, coco superamide, lauric DEA, oleamide DEA, ricinoleic acid alkanolamide, coconut diethanolamide, coco monoethanolamide, lauric diethanolamide, fatty acid alkanolamide, cocoamode DEA, fatty acid alkanolamide, lauric monoethanolamid, lauramide, coconut diethanolamide, coconut monoethanolamide, and combinations thereof.

Exemplary amine oxides include, for example, N,N-dimethyldodecyl amine oxide, N,N-dimethylmyristal amine oxide, cetyl dimethyl amine oxide, alkyl dimethylamine oxide, fatty acid amidoalkyl dimethyl a mine oxide, lauryl dimethyl amine oxide, Bis(2-hydroxy ethyl) cocoamino oxide, myristyl dimethyl amine oxide, isostaramidopropyl morpholine oxide, coco amine oxide, lauryl myristyl dimethyl amine oxide, and combinations thereof.

In another embodiment, the foam stabilizing agent may comprise an electrolyte. In particular, electrolytes may help stabilize foams formed from ionic (e.g., anionic, cationic, amphoteric) foam forming surfactants. Without wishing to be bound by any particular theory, it is believed that increased stabilization may result from the cationic-anionic type interactions between an electrolyte and, for example, an anionic surfactant. Examples of suitable electrolytes include disodium hydrogen phosphate, tetrasodium pyrophosphate, pentasodium tripolyphosphate, and the like.

In another embodiment, the foam stabilizing agent may comprise finely divided particles, e.g., small solid particulates. Such particles may help stabilize foam by preventing the coalescence of bubbles in the foam. Examples of finely divided particles include powdered silica, ferric oxide, and the like.

The foam stabilizing agent may also optionally be a foam stabilizing polymer. Such polymers may stabilize foams by increasing either surface or bulk viscosity of the foam, resulting in increased film elasticity and decreased drainage of liquid from the foam. In particular, the foam stabilizing polymer may be any suitable polyacrylic acid polymer having a molecular weight of from about 10,000 to about 500,000, any suitable ethylene oxide/propylene oxide block copolymer having a molecular weight up to about 30,000, any suitable polyethylene glycol having a molecular weight of 400 or greater, or any suitable biopolymer having a molecular weight of about 100,000 or more, preferably about 1,000,000 or more.

Any suitable polyacrylic and polymer may be used. Polyacrylic acid polymers are commercially available from a variety of sources.

The ethylene oxide/propylene oxide block copolymers are also commercially available polymers. As an example of such polymers, there can be mentioned ANTAROX polymers, such as ANTAROX F-88 block copolymer from Rhodia, Inc.

In other embodiments, the foam stabilizing agent may comprise biopolymers, which act to stabilize the air-liquid interface in the foam. The biopolymer may be a water dispersible or soluble hydrophilic colloid selected form the group consisting of gum ghatti, gum arabic, gum tragacanth, locust bean gum, gum karaya, guar gum, carrageenan, algin, biopolymers such as xanthan gum and welan gum and the hydroxyethyl, carboxymethyl, hydroxyethyl carboxymethyl and hydroxypropyl ether derivatives thereof, and mixtures thereof. These polymers may be used unmodified, as normally isolated from their source materials, or they may be modified as is well known in the polymer art such as by hydroxyalkylation, carboxyalkylation, or mixed hydroxyalkylation carboxyalkylation. Specific examples of modified polymers are carboxymethyl-2-hydroxy-propylpropyl-ether guar gum and 2-hydroxy-3-(trimethyl ammonio) propyl-ether chloride guar gum. Preferred biopolymers for use in this invention are guar and xanthan gums and derivatives thereof. Many of these derivatives form clear solutions in water. Thus, the term water-soluble polymer is intended to mean polymers which form colloidal solutions or colloidal dispersions in water.

The sources of these gums are well known. Gum ghatti is derived from an exudate of the Anogeissus latifolia tree of the Combretaccae family. Gum arabic is derived from an exudate of the acacia tree. Gum tragacanth is derived from an exudate of various species of shrubs belonging to the genus Astragalus. Gum karaya is derived form an exudate of the Sterculia ureus tree. Locust bean gum is derived form the fruit of the carbo tree (Ceratonia siligua). Guar gum is derived form the seeds of two annual leguminous plants, Cyamopsis tetragonalobus and Cyamopsis psoraloides. Algin is derived from all brown seaweeds, of the family Phaeophyceae, although principally from the giant kelp Macrosystis pyrifera. Carrageenan is derived form certain species of red seaweeds of the Gigartinaceae, Solieriaceae, Phylophoraceae, and Hypneaceae families.

In other embodiments, the foam stabilizing agent may be liquid crystalline phases, which may act to enhance the viscosity of the liquid in the foam structure, thus slowing the drainage rate of liquid from the foam.

In one particularly preferred embodiment, the foam stabilizing agent is an acyl lactylate. Acyl lactylates suitable for use as a foam stabilizing agent have the general structural formula:

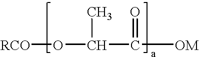

wherein RCO radical is a C₆ to C₁₄ acyl radical; a is a number from 1 to 3, and typically 1 to 2; M is an alkali metal, an alkaline earth metal, ammonium, or a substituted ammonium group having one or more C₁ to C₃ alkyl or hydroxyalkyl groups. The preferred RCO acyl radicals are C₈ to C₁₂ acyl radicals, and the preferred M is ammonium or an alkali metal.

Acyl lactylates are described in various patents, for example, U.S. Pat. No. 5,911,981, herein incorporated by reference. Various acyl lactylates also are available commercially. Commercial acyl lactylates typically are a blend of the monolactylate (i.e., a=1) and the dilactylate (i.e., a=2), and contain small amounts of trilactylate (i.e., a=3). As used herein, the term “acyl lactylate” is defined as either a pure monolactylate, a pure dilactylate, a pure trilactylate, or a commercial mixture containing the mono-, di-, and trilactylates.

Examples of acyl lactylates having the above general structure include, but are not limited to, sodium lauroyl monolactylate, sodium myristoyl monolactylate, sodium decanoyl monolactylate, potassium dodecanoyl monolactylate, potassium dodecanoyl dilactylate, sodium myristoyl dilactylate, sodium lauroyl dilactylate, triethanolammonium (TEA) dodecanoyl monolactylate, ammonium decanoyl monolactylate, triethanolammonium decanoyl monolactylate, sodium caprooyl lactylate, sodium capryloyl lactylate, sodium caproyl lactylate, sodium cocoyl lactylate, sodium lauroyl lactylate, monoethanolammonium (MEA) lauroyl lactylate, and potassium lauroyl lactylate.

For illustrative purposes, the following structures depict sodium caproyl lactylate and sodium lauroyl lactylate, respectively:

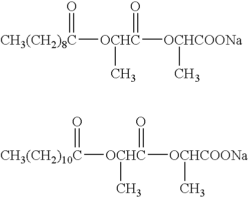

As noted above, the foam stabilizing agent may be a viscosity enhancing agent. Viscosity enhancing agents act to thicken, gel, or harden the foam barrier or interceptor structure such that the foam is immobilized or locked at the location (e.g., on the portion of the absorbent article) at which it forms. The viscosity enhancing agent is especially useful in minimizing the migration and loss of the foam barrier or interceptor structure.

Exemplary viscosity enhancing agents include organic materials such as natural or synthetic waxes, C₁₂-C₆₀ alcohols, C₁₂-C₆₀ acids, alpha-hydroxy fatty acids, polyhydroxy fatty acid esters, polyhydroxy fatty acid amides, and inorganic/organic materials such as metal ester complexes containing zinc, calcium, aluminum or magnesium, fumed silicas, and organoclays.

Suitable viscosity enhancing agents include C₁₂-C₆₀ alcohols, preferably C₁₆-C₂₂ fatty alcohols, most preferably crystalline high melting materials selected from the group consisting of cetyl alcohol, stearyl alcohol, behenyl alcohol, and mixtures thereof. Behenyl alcohol is particularly preferred.

Other suitable viscosity enhancing agents include C₁₂-C₆₀ acids, preferably C₁₆-C₂₂ fatty acids, most preferably selected from the group consisting of palmitic acid, stearic acid, behenic acid, oleic acid, linoleic acid, myristic acid, ricinoleic acid, eurcic acid, lauric acid, isostearic acid, and mixtures thereof. Mixtures of palmitic acid and stearic acid are particularly preferred. The substantially linear structure and high melting point of these viscosity enhancing agents speed up the solidification of the composition and lock it to the location on the delivery vehicle where it is deposited. Other suitable viscosity enhancing agents that may be used herein are alpha-hydroxy fatty acids, including 12-hydroxystearic acid, 12-hydroxylauric acid, 16-hydroxyhexadecanoic acid, and mixtures thereof.

Waxes are also suitable for use herein as viscosity enhancing agents. Natural waxes may include, but are not limited to, carnauba, ozokerite, beeswax, candelilla, paraffin, ceresin, esparto, ouricuri, rezowax, and other known mined and mineral waxes. Synthetic waxes may include, but are not limited to, paraffin waxes and microcrystalline waxes. It is particularly advantageous to use these waxes that form microcrystalline structures upon cooling and/or solidifying.

Also suitable for use herein as viscosity enhancing agents are block copolymers made from styrene and olefinin monomers. Particularly preferred block copolymers for use herein are polystyrene-ethylene/butylene-polystyrene block copolymers and linear block copolymers of styrene-ethylene/butylene-styrene (S-EB-S). Also preferred for use herein are polystyrene-ethylene-ethylene/propylene-styrene (S-E-EP-S) block copolymers, wherein the ethylene/propylene block is derived from selective hydrogenation of the unsaturation sites within the polystyrene-isoprene/butadiene-styrene block copolymers.

Other suitable viscosity enhancing agents that may be used herein include polyhydroxy fatty acid esters, polyhydroxy fatty acid amides, and mixtures thereof. Preferred esters and amides will have three or more free hydroxy groups on the polyhydroxy moiety and are typically nonionic in character. Because of the possible skin sensitivity of those using articles to which the composition is applied, these esters and amides should also be relatively mild and non-irritating to the skin.

Other viscosity enhancing agents useful in the present invention include metal ester complexes of aluminum, magnesium, or zinc with stearates, benehates, palmitates or laureates. Other examples of suitable viscosity enhancing agents include alkyl galactomannan, talc, magnesium silicate, sorbitol, colloidal silicone dioxide, magnesium aluminum silicate, wool wax alcohol, sorbiton, sesquioleate, cetyl hydroxylethyl cellulose, and other modified celluloses.

### Skin Feel Enhancing Agents and Surface Enhancing Agents

Typically, the pre-use foam composition is located on or near the topsheet of the absorbent article, to ensure the foam structure formed when the pre-use foam composition is contacted with the triggering agent will be formed on the topsheet. Thus, in certain embodiments, the pre-use foam composition may come in contact with the user's body before activation by the triggering agent. It is thus desirable that the pre-use foam composition be non-irritating and have good skin feel properties. Thus, in certain embodiments, the pre-use foam composition may also further comprise a skin feel enhancing agent to improve the feel of the pre-use foam composition on the skin of the user. Advantageously, the skin feel enhancing agent may also be incorporated into the foam barrier/interceptor structure formed when the pre-use foam composition is contacted with the triggering agent, and may thus also act to improve the skin feel properties of the foam structure. It is thus desirable that both the pre-use foam composition and the foam barrier/interceptor structure have good skin feel properties. In particular, it is preferable that the pre-use foam composition be smooth and non-irritating to the skin of the wearer, and the foam structure preferably be non greasy, sticky, or tacky when contacting the skin.

Thus, in certain embodiments, the foam forming adjuvant may further comprise additional agents that act to improve the skin feel properties of the pre-use foam composition and/or the foam barrier/interceptor structure. As used herein, “skin feel enhancing agent” thus refers to an agent that creates or modifies the feel of the pre-use foam composition and/or the foam barrier/interceptor structure on the skin.

Examples of suitable skin feel enhancing agents are known in the art and include, for example, polymeric skin feel aids such as those described in U.S. Pat. No. 5,002,680, herein incorporated by reference, and particulates like microbeads, such as those described in U.S. Pat. No. 7,163,669, herein incorporated by reference.

Preferably, the pre-use foam composition comprises skin feel enhancing agents in an amount of from about 0.001% by weight to about 30% by weight of the composition, more preferably from about 0.001% by weight to about 5% by weight of the composition.

Optionally, the foam forming adjuvant may further comprise surface enhancing agents, such as anti-adherence agents, adherence agents, and combinations thereof.

For instance, it is generally preferable that the foam barrier or interceptor structures have minimal adherence to the body of the user upon removal of the absorbent article to avoid unwanted foam residue sticking to the skin of the user and a resulting messy clean up. Thus, in one embodiment, the pre-use foam composition may further comprise at least one anti-adherence agent. An anti-adherence agent may act by a variety of mechanisms. For instance, an anti-adherence agent may deposit on the skin surface to act as a release agent, may reduce surface interactions between the foam barrier/interceptor structure and skin, may remove materials such as bodily exudates (e.g., menses) from the skin surface that might increase adherence, and the like.

Anti-adherence agents suitable for use in the absorbent articles described herein are described in, for example, U.S. Patent App. No. 2006/0140899, herein incorporated by reference. Other examples of suitable anti-adherence agents include silicone release agents, as well as viscoelastants, such as those described in U.S. Pat. No. 6,060,636, herein incorporated by reference.

The amount of anti-adherence agents incorporated into the pre-use foam composition will vary depending on the type of foam and its placement within the absorbent article, but typically will be from about 0.1% to about 5% by weight of the composition.

As noted above, the foam forming adjuvant may further comprise an adherence agent. Advantageously, adherence agents may be used to adhere the foam barrier structure to the topsheet of the absorbent article to prevent or reduce migration of the foam barrier structure across the surface of the topsheet, which may otherwise occur as a result of friction created by movement of the user.

Any adhesive suitable for contact with skin may be used as an adherence agent. Examples of suitable adherence agents include pressure sensitive adhesives, spray adhesives, hot-melt adhesives, self-adhering elastomeric materials, and the like. Specific examples of suitable adhesives include polysiloxanes, polyacrylates, polyurethanes, tacky rubbers such as polyisobutylene, and the like. Other suitable adhesives include elastomeric block copolymers, such as those described in WO 95/01408, herein incorporated by reference.

The amount of adherence agents incorporated into the pre-use foam composition typically will be from about 0.1% to about 5% by weight of the composition.

### Trigger Control Agents

Optionally, the foam forming adjuvant may be a trigger control agent. As used herein, the term trigger control agent refers to compounds that slow the triggering process. For example, in some embodiments, the gas providing agent may be activated by contact with water present in a triggering agent, such as urine. A trigger control agent may be incorporated into the absorbent article to slow the diffusion of a body exudate into the pre-use foam composition. In this manner, the extent and speed of foaming can be controlled.

Examples of suitable trigger control agents include, for example, oleophilic compounds, such as oleophilic waxes or polymers. Examples of oleophilic waxes include carnauba wax, beeswax, candlewax, and the like. Examples of oleophilic polymers include poly(N-vinylpyrrolidone), polyacrylates (e.g., polymethylmethacrylate, poly-n-butylmethacrylate), cellulose esters (e.g., cellulose acetate butyrate, cellulose acetate proprionate), poly(vinyl alcohol) and the like.

Other examples of suitable trigger control agents include decelerants and accelerants. Decelerants act by reducing the solubility rate of components in the pre-use foam composition, thereby decreasing the speed at which the foam barrier/interceptor structure is formed, while accelerants act by increasing the solubility rate of components in the pre-use foam composition, thereby increasing the speed at which the foam barrier/interceptor structure is formed.

The amount of trigger control agent in the pre-use foam composition is typically from about 0.001% to about 5% by weight.

### Hair and Skin Care Agents

The foam forming adjuvant may optionally comprise a hair and/or skin care agent, such as an emollient. Examples of suitable hair and skin care agents are described in, for example, U.S. Patent App. Publ. No. 2003/0082129, herein incorporated by reference. Preferably, the hair or skin care agents do not destabilize the foam barrier/interceptor, such as those listed in U.S. Patent App. Publ. No. 2007/0134167, herein incorporated by reference.

The hair or skin care agents may provide skin benefits or to assist in achieving desirable foam properties. For instance, in one embodiment, the pre-use foam composition comprises components that aid in skin care. Preferably, such agents include those that have been deemed safe and effective skin care agents. Such materials include Category I actives as defined by the U.S. Federal Food and Drug Administration's (FDA) Tentative Final Monograph on Skin Protectant Drug Products for Over-the-Counter Human Use, which presently include: alantoin, aluminum hydroxide gel, calamine, cocoa butter, dimethicone, cod liver oil (in combination), glycerine, kaolin, petrolatum, lanolin, mineral oil, shark liver oil, white petrolatum, talc, topical starch, zinc acetate, zinc carbonate, zinc oxide, and the like. Other potentially useful materials are Category III actives as defined by the U.S. Federal Food and Drug Administration's Tentative Final Monograph on Skin Protectant Drug Products for Over-the-Counter Human Use tentative final monograph on skin protectant drug products for over-thecounter human use, which presently include: live yeast cell derivatives, aldioxa, aluminum acetate, microporous cellulose, cholecalciferol, colloidal oatmeal, cysteine hydrochloride, dexpanthanol, Peruvian balsam oil, protein hydrolysates, racemethionine, sodium bicarbonate, Vitamin A, and the like.

The pre-use foam compositions can optionally comprise other components typically present in emulsions, creams, ointment, lotions, powders, suspensions, and the like. These components include viscosity modifiers, perfumes, disinfectant antibacterial actives, antiviral agents, vitamins, pharmaceutical actives, film formers, deodorants, opacifiers, astringents, solvents, preservatives, and the like. In addition, stabilizers can be added to enhance the shelf life of the composition such as cellulose derivatives, proteins and lecithin. All of these materials are well known in the art as additives for such formulations and can be employed in appropriate amounts in the compositions for use herein.

Emollients may be used as skin care agents and may be in the form of natural or synthetic esters, silicone oils, hydrocarbons, starches, fatty acids and mixtures thereof. Typically the emollient may range in concentration from about 0.1% to about 35% by weight of the pre-use foam composition, and more preferably from about 0.1% to about 10% by weight of the pre-use foam composition.

Silicone oils may be divided into the volatile and nonvolatile variety. The term “volatile” as used herein refers to those materials which have a measurable vapor pressure at ambient temperature. Volatile silicone oils are preferably chosen from cyclic or linear polydimethylsiloxanes containing from 3 to 9, preferably from 4 to 5, silicon atoms.

Linear volatile silicone materials generally have viscosities less than about 5 centistokes at 25° C. while cyclic materials typically have viscosities of less than about 10 centistokes.

Nonvolatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 100,000 centistokes at 25° C. Among the preferred non-volatile emollients useful in the present compositions are the polydimethyl siloxanes having viscosities from about 10 to about 400 centistokes at 25° C.

Exemplary ester emollients include:

(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.

(2) Ether-esters such as fatty acid esters of ethoxylated fatty alcohols.

(3) Polyhydric alcohol esters. Ethylene glycol mono and di-fatty acid ester, diethylene glycol mono- and di-fatty acid esters, polyethylene glycol (200-6000) mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.

(4) Wax esters such as beeswax, spermaceti, myristyl myristate, stearyl stearate and arachidyl behenate.

(5) Sterols esters, of which cholesterol fatty acid esters are examples thereof.

(6) Triglycerides such as sunflower seed oil, maleated sunflower seed oil, borage seed oil and safflower oil.

Hydrocarbons suitable as emollients include petrolatum, mineral oil, isoparaffins and hydrocarbon waxes such as polyethylene.

Starches are also suitable emollients. Typical of this class is tapioca and arabinogalactan.

Fatty acids may also be suitable as emollients. The fatty acids normally have from 10 to 30 carbon atoms. Illustrative of this category are pelargonic, lauric, myristic, palmitic, stearic, isostearic, hydroxystearic, oleic, linoleic, riconleic, arachidic, behenic and erucic acids.

Particularly preferred emollients include those that may act as both a foam forming agent and a foam stabilizing agent, such as various surfactants, fatty acids, and the like.

### Film-Forming Agents

Optionally, the foam forming adjuvant may also comprise film-forming agents. The film-forming agents enhance the adherence of the foam barrier structure to the skin, improving the barrier function of the foam.

Useful herein as film-forming agents are polyalkenes including polyethylenes having a molecular weight ranging from about 300 to about 3000; polyisobutylenes; polyisobutenes; polydecenes; and hydrogenated polyisobutenes.

Other suitable oleophilic film-forming agents are copolymers of vinylpyrrolidone (PVP) and long chain alpha olefins, including, but are not limited to, PVP/eicosene copolymers, and tricontanyl PVP copolymers.

Also suitable for use herein as film-forming agents are acrylic copolymers having long (C₈-C₃₀) alkyl chains to enhance their oleophilicity, such as acrylate/octylacrylamide copolymers. Other suitable film-forming agents include, but are not limited to, polyethylene glycol derivatives of Beeswax; and fatty acid ester/fatty acid anhydride grafted polyolefins wherein the esters and anhydrides are derived from C₁₂-C₂₂ fatty acid moieties, for example, C₃₀-C₃₈ olefin/isopropyl maleate/maleic anhydride copolymer.

### Liquids

Optionally, the pre-use foam composition may further comprise water or some other suitable liquid that may be incorporated into the foam barrier/interceptor structure formed when the triggering agent contacts the pre-use foam composition. As noted above, the foam barrier/interceptor structure is desirably a wet liquid foam structure. In certain embodiments, liquid incorporated into the wet foam structure may be provided by the triggering agent itself. For instance, the triggering agent may be a body exudate such as urine, menses, sweat, vaginal secretions, feces, and combinations thereof. Upon contact with the pre-use foam composition, moisture present in the body exudate may interact with the pre-use foam composition and be incorporated into the wet foam barrier/interceptor structure.

In other embodiments, it may be desirable to incorporate liquids into the pre-use foam composition itself. For example, the pre-use foam composition may comprise encapsulated water or other liquids. Upon contact with the triggering agent, the shell encapsulating the liquid may be dissolved or otherwise ruptured, releasing the liquid which then interacts with the other components of the pre-use foam composition to form the foam barrier/interceptor structure.

By incorporating liquid into the pre-use foam composition, it is not necessary for the triggering agent to comprise a liquid. This is particularly advantageous in instances where the triggering agent is not a body exudate, but rather, is another trigger such as body heat or pressure. Incorporation of a liquid into the pre-use foam composition is particularly beneficial when the triggering agent is the result of an action by the article user, such as removal of a release strip on the absorbent article. In this embodiment, removal of the release strip may rupture the encapsulant encasing the liquid, releasing the liquid and allowing for formation of the foam barrier/interceptor structure. Encapsulated liquid may also be included in the pre-use foam composition in instances where the triggering agent is a body exudate and itself comprises a liquid. Means for incorporating liquids into absorbent articles are described in U.S. Pat. Nos. 6,695,828 and 6,666,850, as well as U.S. Publ. Pat. App. No. 2007/0072780, all herein incorporated by reference.

### Other Optional Components

Optionally, the foam forming adjuvant may further comprise preservatives. Examples of suitable preservatives include, for example, propyl paraben, methyl paraben, benzyl alcohol, benzalkonium, tribasic calcium phosphate, BHT, and the like. Other examples of suitable preservatives include acids such as citric acid, tartaric acid, maleic acid, lactic acid, malic acid, benzoic acid, salicylic acid, and the like.

Typically, the pre-use foam compositions of the present disclosure are not simply introduced into or onto the absorbent article without a stabilizing mechanism to ensure the composition stays in the desired area. The pre-use foam composition of the present disclosure may be introduced into or onto the topsheet, absorbent core, or another layer of the absorbent article utilizing various methods including, for example, spray coating, slot coating and printing, or a combination thereof. With spray coating, the pre-use foam composition is first thoroughly mixed with an agent that acts to stabilize the pre-use foam composition, usually an adhesive, such as a urine-soluble adhesive agent to disperse the composition throughout the adhesive material. It will be recognized that if the composition comprises separate compounds such as an acid and a base or a mixture of acids and a mixture of bases to be utilized in neat form, care should be taken to ensure the adhesive is non-reactive with the acid and/or base or mixtures to prevent premature activation of the gas providing agent. Further, when and acid/base mixture is utilized as the gas providing agent, typically the acid is separately mixed with an adhesive and the base is separately mixed with an adhesive and the respective mixtures are applied in separate layers on the substrate.

The adhesive material can optionally comprise an exudate-soluble adhesive which will partially or completely dissolve upon, e.g., urination by the wearer and allow the urine or other exudate to contact the pre-use foam composition. Although discussed herein primarily in terms of a triggering agent that is urine, it should be understood that the adhesive may partially or completely dissolve upon contact with liquid present in other triggering agents, such as menses, feces, vaginal secretions, sweat, and the like. Suitable urine-soluble adhesives include, for example, polyvinyl pyrrolidone and polyvinyl alcohol, gums, alginates, and combinations thereof. Alternately, the adhesive may be a non-urine soluble adhesive. After the adhesive and pre-use foaming composition are thoroughly mixed, they can be applied onto the desired area of the absorbent article by spray coating, knifing, or roller coating, for example, and allowed to dry. The adhesive/pre-use foaming composition mixture adheres to the topsheet, absorbent core, or another layer where it is retained until wetted by the triggering agent. Upon wetting, the adhesive releases the components of the pre-use foam composition such that the gas providing agent is activated and releases or produces gas that combines with the foam forming agent and other components of the pre-use foam composition to produce the foam barrier or interceptor structure. Typically, the pre-use foam composition/adhesive mixture comprises from about 5% to about 99% pre-use foam composition by weight, preferably from about 50% to about 99% pre-use foam composition by weight. It will be recognized by one skilled in the art that the mixture ratio of the pre-use foam composition and adhesive may vary depending upon the material construction upon which the mixture is sprayed. In a particular embodiment, a first layer comprising a pre-use foam composition and an adhesive are sprayed onto the substrate. After the first layer has dried, a second layer comprising an adhesive agent is sprayed on top of the first layer. This combination of two layers may help ensure that the pre-use foam composition does not foam prior to contact with a triggering agent.

Similar to spray coating, the pre-use foam composition may be introduced into or onto the absorbent article through slot coating. In slot coating, an adhesive/pre-use foam composition mixture as discussed above is introduced directly onto the desired area of the absorbent article in “slots” or discrete row patterns. Upon contact with a triggering agent, the adhesive allows a release of the pre-use foam composition such that the gas providing agent may produce a gas and a foam barrier or interceptor structure may form. Slot coating may be advantageous in certain applications where it is not desirable to coat the entire surface with an adhesive. In some circumstances, an adhesive coating over an entire surface may retard quick absorption of the body exudate into the absorbent core. When slot coating is utilized, channels are created where no adhesive is present and the body exudate may drain quickly. Slot coating may also be advantageous in certain applications where precise control of the location of the pre-use foam composition is desired. Typically, the rows of pre-use foam composition/adhesive are spaced on the order of from about 0.1 inches to about 3 inches apart from each other, preferably from about 0.2 inches to about 2 inches apart from each other, and most preferably about 0.25 inches apart from each other. Generally, the rows are evenly spaced across the surface upon which they are applied, but may be spaced in specific patterns with varying spacing if desired, and as discussed elsewhere herein. In a particular embodiment utilizing a gas providing agent comprised of an acid/base mixture, slot coating can be utilized to create alternating rows of acid/adhesive—base/adhesive to ensure that the acid and base do not react together prior to contact with the triggering agent. As described above in regard to spray coating, a second layer of adhesive may be introduced on top of a first slot-coated layer comprising both the pre-use foam composition and an adhesive to further ensure that the gas providing agent does not release gas prior to urination.

The pre-use foam composition can also be introduced onto or into a gas-permeable topsheet, absorbent core, or another layer of the absorbent article through the use of a vacuum driving force. The pre-use foam composition is positioned on the liner, absorbent core, or another layer while a vacuum driving force is applied to the opposite side of the topsheet, core, or layer to drive the foam composition into the fabric matrix of the tophseet, core, or other layer. Varying degrees of vacuum can be applied depending upon the depth the pre-use foam composition is to be positioned within the substrate. In this embodiment, no urine-soluble adhesive is necessary. Once in the fabric matrix of the pad, the pre-use foam composition is retained until contact with a triggering agent occurs at which time the triggering agent contacts the pre-use foam composition and a foam barrier or interceptor structure is produced. This embodiment is particularly useful when the gas providing agent, foam forming agent, or other component of the pre-use foam composition is solid or powdered and applied neat. Alternatively, electrostatic forces or other means may be utilized to stabilize the pre-use foam composition on the surface of the topsheet or core.

Absorbent articles comprising a pre-use foam composition can be produced by forming a solution and/or suspension from the foam forming agent(s), the adhesive, the gas providing agents and/or any desired additional components of the pre-use foam composition, as discussed herein, using water and/or polar and/or nonpolar solvents. The substrate to be coated, e.g., the topsheet, absorbent core, etc. of the absorbent article, is then coated or impregnated with the solution or suspension and the support thus treated is dried.

If all the components of the pre-use foam composition are to be arranged as a single mixture of components on the support, a suspension thereof may be formed in a nonpolar solvent. The support is then dipped into such suspension or otherwise impregnated therewith, after which the solvent is evaporated.

When the application is effected through two separate mixtures of components, separate solutions or suspensions are formed in water and/or polar and/or nonpolar solvents. For instance, one of the mixtures of components may contain an acid gas providing agent, while the other contains a base gas providing agent. Further, one or both of such mixtures of components may contain the foam forming agent(s) and, possibly, further components of the pre-use foam composition. The solutions or suspensions thus produced are applied to the support separately in the form of adjacent strips, spots or the like. For example, the topsheet of the absorbent article may be impregnated on both sides with the solutions or suspension, in which connection the depth of penetration is selected such that the two mixtures of components do not contact each other.

Further, supports consisting, e.g., of a paper-fiber fleece or non-woven fabric, may in each case be treated with one of the mixtures of components, dried and then joined at the faces to each other so as to form a unit, by means of a joining layer, which may likewise consist of the same support material and which possesses an adhesive layer.

In an alternative embodiment, the pre-use foam composition may be incorporated into a hydrophilic or hydrophobic microsponge material which is subsequently used in combination with the absorbent article of the present disclosure. The microsponge performs the same function as the microencapsulation shell described above. The hydrophilic microsponge containing the pre-use foam composition is introduced onto or into the absorbent article as discussed above. Suitable materials comprising the hydrophilic microsponge include, for example, acrylate polymers and acrylate copolymers of a hydrophilic nature.

To incorporate either solid or liquid components of the pre-use foam composition into the hydrophilic microsponge material, the components can be introduced into a suitable volatile, such as an alcohol or water and dissolved. In instances where the gas providing agent comprises an acid and a base, the acid and/or base may be dissolved in separate solvents to prevent premature production of gas. The solution containing the dissolved acid and/or base (and other components) is contacted with and absorbed into the microsponge material. After absorption, the solvent is driven off by evaporation or other means known in the art, leaving a dry hydrophilic microsponge material containing the pre-use foam composition components. Because of the structure of the sponge, the gas providing agent may need to be suitably stable as it would need to travel a tortuous path to exit the sponge. The gas providing agent is located in the interstitial spaces in the sponge and, upon flooding of the sponge upon urination or contact with other body exudates, is driven out of the interstitial spaces by the exudate and releases a gas. The pre-use foam composition-containing hydrophilic microsponges of the present invention may be introduced onto or into the absorbent articles in a manner similar to the encapsulated agents discussed above.

The delivery configuration and chemical composition of the pre-use foam composition controls both where and when the barrier/interceptor is formed, and may be determined based on the form of the benefit desired. For instance, in certain embodiments, it may be desirable for the foam barrier or interceptor structure to quickly form upon contact with the triggering agent, while in other instances, it may be desirable for the foam barrier or interceptor structure to form more slowly.

As noted above, the speed at which the foam structure is formed upon contact with the triggering agent may be controlled in part by the types of agents that comprise the pre-use foam composition. For instance, the speed at which the foam barrier or interceptor structure is produced may be slowed by using a gas providing agent that has been microencapsulated and/or that comprises a solid material containing an entrapped gas that is slowly released as the solid material is dissolved. Other agents, such as the trigger control agents described herein, may also be incorporated into the absorbent article to slow the contact of the triggering agent with the pre-use foam composition.

Additionally, the placement of the pre-use foam composition in the absorbent article will affect where and when the barrier/interceptor is formed. As noted herein, the pre-use foam composition may be positioned into any suitable layer of the absorbent article including, for example, the topsheet and/or absorbent core, among others. It is generally preferable, however, that the pre-use foam composition be incorporated into the absorbent article in such a manner that a foam will be produced on the bodyfacing side of the topsheet of the absorbent article upon contact of the pre-use foam composition with the triggering agent. By positioning the composition such that the foam is produced on the topsheet, the foam can expand on top of the article, either filling the void between the topsheet and the user's body to create a comfortable barrier or interceptor that conforms to the user's body during use of the article, or expanding sufficiently to form a raised barrier structure on the body-facing surface of the topsheet.

The components of the pre-use foam composition, such as the gas providing agent, the foam forming agent, the foam stabilizing agent, and any additional components, may be mixed together and applied to the same portion of the absorbent article, e.g., as a layer or strip of pre-use foam composition. Alternately, the components of the pre-use foam composition may be applied separately to the absorbent article. For instance, in one embodiment, the gas providing agent may be applied first, and may optionally be applied with an adhesive to hold the gas providing agent in place, with the foam forming agent then being applied on top of the gas providing agent-containing layer, and other composition components being included with the gas providing agent layer, foam forming agent layer, or applied as additional layers. Alternately, the foam forming agent may be applied first, with the gas providing agent applied on top of the foam forming agent-containing layer, and other composition components being included with the gas providing agent layer, foam forming agent layer, or applied as additional layers.

In one particular embodiment, the pre-use foam composition is incorporated into or onto the topsheet of the absorbent article. The composition may be applied to the body-facing surface of the topsheet, to the outer-facing surface of the topsheet, or to both the body-facing and outer-facing surfaces of the topsheet. In one specific embodiment, the pre-use foam composition is applied uniformly over the entire surface of one side of the topsheet of an absorbent article. The composition may be applied using any suitable method, such as those described herein. The triggering agent, such as urine, menses, or other bodily exudate, may pass through the layers of pre-use foam composition, wetting the composition, resulting in release of gas from the gas providing agent, which causes the foam forming agent to foam.

In still other embodiments, components of the pre-use foam composition may be applied to different portions of the absorbent article. For instance, in one embodiment, the gas providing agent may be applied to one portion of the absorbent article and the foam forming agent applied to a separate portion of the absorbent article, with any other additional composition components being applied with either the gas providing agent, foam forming agent, or optionally to still different portions of the absorbent article. The gas providing agent and foam forming agent should, however, be applied in close enough proximity that the gas released from the gas providing agent will cause the foam forming agent to foam upon contact with a triggering agent.

In one particular embodiment, the absorbent article comprises bands of the pre-use foam composition deposited on the topsheet along at least a portion of the periphery of the article. The band may have a laterally inboard dimension generally overlying the absorbent structure and, in one embodiment, may have a laterally outboard dimension having the same general shape as the underlying absorbent structure so as to be generally coextensive with a periphery of the absorbent structure. In an alternate embodiment, the bands may extend laterally outboard to the edges of the article. The bands may be placed in any number of deposited patterns. When the bands of pre-use foam composition are contacted with a triggering agent, a foam barrier is formed along at least a portion of the periphery of the article, which acts as a barrier to lateral flow of bodily fluids from the underlying absorbent or fluid that flows across the top cover before being absorbed by the absorbent structure.

The pre-use foam composition may be applied in various patterns within the band or, alternately, the band can comprise one continuous deposit of pre-use foam composition. In one particular embodiment, the deposit pattern may be defined by a series of continuous deposits of the composition, for example, a band of relatively thin spaced apart stripes. Each stripe may comprise all components of the pre-use foam composition (e.g., the foam forming agent, gas providing agent, foam stabilizing agent, and any additional components), or alternately, stripes comprising different components of the pre-use foam composition may be deposited side by side (e.g., a band of gas providing agent next to a band of foam forming agent).

The band may have an overall width dimension overlying the absorbent structure of between about 5-20 mm. In embodiments where the band is defined by a series of thin spaced apart stripes, each stripe may have a width of about 0.25 mm, and may be spaced apart from about 0.25 mm to about 0.75 mm. Various other continuous deposit patterns are also within the scope of the disclosure. The pattern of continuous stripes may be desired in that a continuous unbroken barrier is defined transverse to the direction of leakage. For example, if the band is defined as a longitudinally extending band along a lateral side margin to prevent leakage from the article sides, it may be desired that the band be generally continuous along the complete length thereof so that the foam structure formed when the triggering agent contacts the band of pre-use foam composition prevents fluid from traveling along the barrier and then migrating through a space in the barrier pattern. In alternate embodiments, the pattern of deposit may be defined by a series of discontinuous deposits of the composition, such as dashed lines, dots, or any other pattern providing sufficient exposed surface area of the top cover.

As noted above, the manner in which the pre-use foam composition is deposited into or onto the absorbent article may affect how the foam barrier structure is formed. For instance, deposit of the pre-use foam composition onto the absorbent article as thinly spaced apart stripes or discontinuous deposit may act to define a tortuous path for certain triggering agents, such as body exudates, slowing the flow of fluid and preventing all of the pre-use foam composition from being triggered at once. In this manner, a foam barrier structure may be continuously formed, as the pre-use foam composition is slowly contacted by the body fluid triggering agent as it migrates towards the lateral sides of the absorbent article.

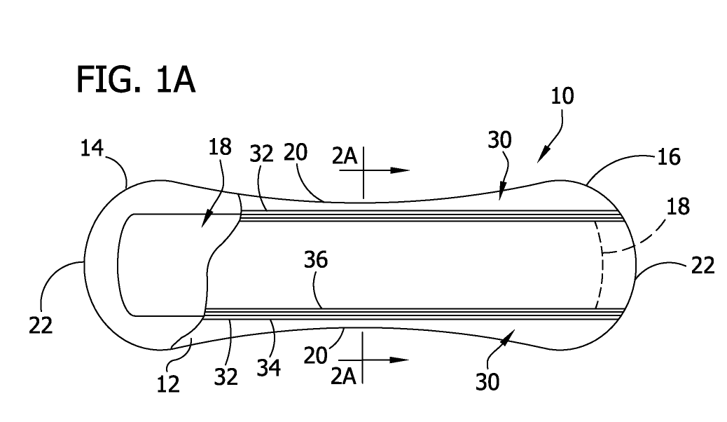

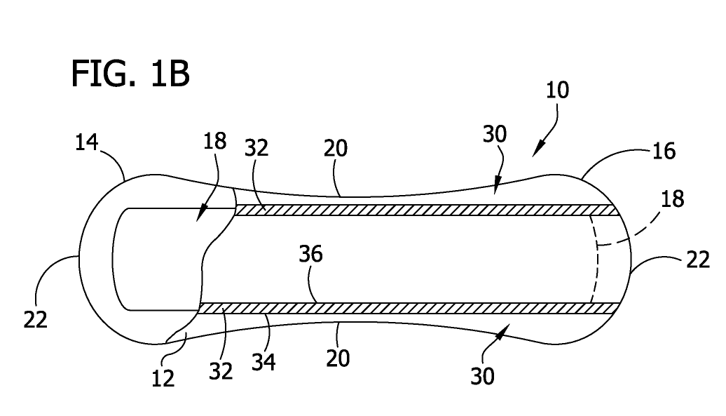

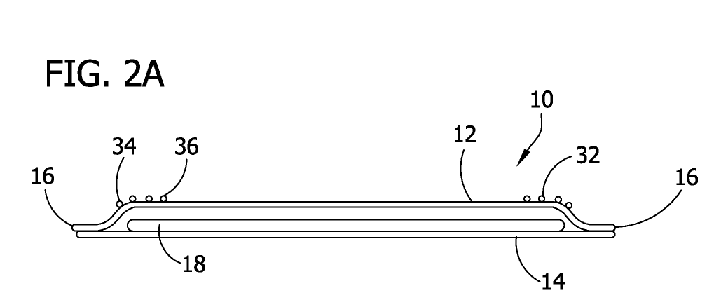

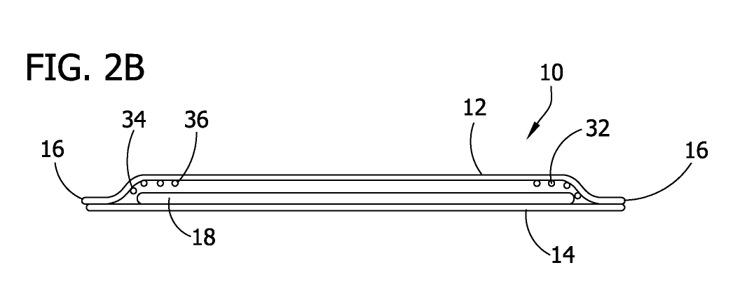

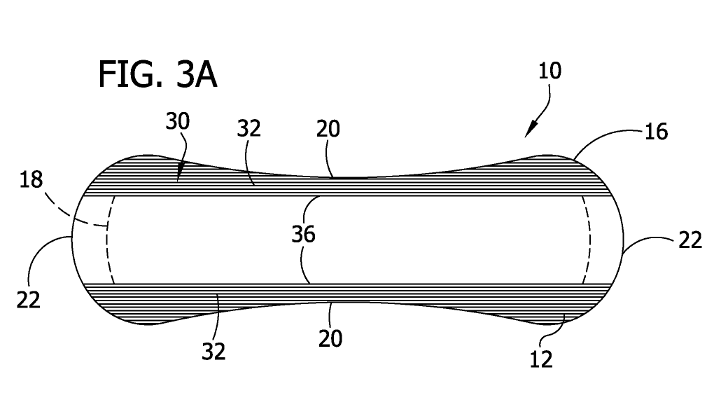

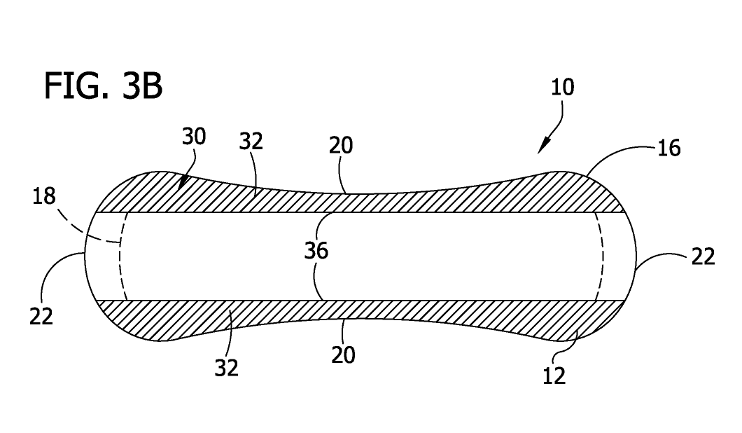

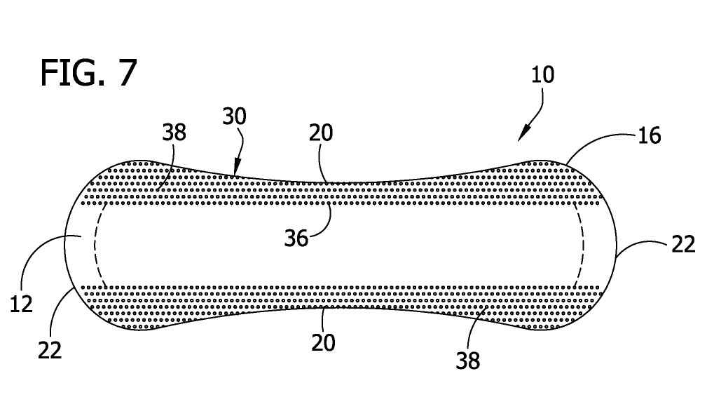

Various embodiments have been illustrated in the figures. Referring generally to FIGS. 1A, 2, 3A, 4A, 5A, and 6-7, embodiments of the absorbent article 10 may include bands 30 of spaced apart deposits 32 of the pre-use foam composition defined on the topsheet 12. The bands 30 may be defined in a longitudinally extending pattern along opposite lateral sides of a longitudinal centerline of the article 10. At least a portion of each band 30 has a laterally inboard dimension 36 overlying a longitudinally extending periphery portion of the underlying absorbent core 18. In certain embodiments for example as illustrated in FIGS. 1, 4, 5, and 6, the bands 30 include a laterally outboard side 34 that is spaced from the sealed peripheral edge 16 of the article 10. In other words, the bands 30 are defined as discrete bands having a generally uniform width along the article 10. In alternate embodiments, for example as illustrated in FIGS. 3 and 7, the bands 30 may extend laterally outward to the sealed peripheral edge 16 of the article 10. It should be appreciated that the bands 30 may be provided on the body-facing surface of the topsheet 12, as shown in FIG. 2A, or may be defined on the inner surface (facing the absorbent core 18) of the topsheet 12, as shown in FIG. 2B. In alternate embodiments, as illustrated in FIGS. 1B, 3B, 4B, and 5B, the absorbent article 10 may comprise a band 30 defined by a single continuous deposit 32 of pre-use foam composition defined on the topsheet 12.

Referring to FIGS. 1 and 2, it can be seen that the article 10 may include two longitudinally extending bands 30 of pre-use foam composition deposits 32. In one embodiment illustrated in FIGS. 1A and 2, the bands 30 are defined by relatively thin, parallel, and continuous stripes extending longitudinally on opposite sides of a centerline axis of the article 10. In an alternate embodiment illustrated in FIG. 1B, the bands 30 are defined by one continuous deposit of pre-use foam composition 32 extending longitudinally on opposite sides of a centerline axis of the article 10. The bands 30 are defined by laterally outboard side 34 and laterally inboard side 36. The laterally outboard side 34 may be generally coextensive with an outer periphery of the underlying absorbent core 18. Thus, in this embodiment, the band completely overlies longitudinal periphery portions or edges of the underlying absorbent core 18. The bands 30 may extend completely between the longitudinal ends 22 of the article 10, as depicted in FIG. 1. In an alternate embodiment as illustrated in FIG. 3, the parallel deposit stripes (FIG. 3A) or single continuous deposit (FIG. 3B) 32 extend laterally outboard to the sealed periphery edges 16 of the article 10.

The individual deposits 32 when in the stripe configuration of FIGS. 1A, 2, and 3A, may have an individual width of about 0.25 mm and be spaced apart by at least the width of one stripe, and desirably at least three stripes. For example, an exposed area of about 0.75 mm may be defined between each parallel stripe, with the band having an overall transverse width between about 5 mm to about 20 mm, preferably between 8 mm to about 14 mm, along the portion overlying the absorbent structure 18. For example, referring to FIG. 1, the width between the laterally outboard side 34 and laterally inboard side 36 may be between about 5 mm to about 20 mm. Referring to FIG. 3, the transverse width of the band 30 between the laterally inboard side 36 to the sealed edges 16 of the article 10 may vary and be substantially greater than 20 mm. However, the portion of the band 30 overlying the absorbent core 18 may be between about 5 mm to about 20 mm, for example from about 8 mm to about 14 mm. It should be appreciated that the transverse width dimension is not a limiting factor and various widths are contemplated and may be empirically determined to provide the benefits of preventing leakage of bodily exudates at the lateral sides 20 of the article 10.

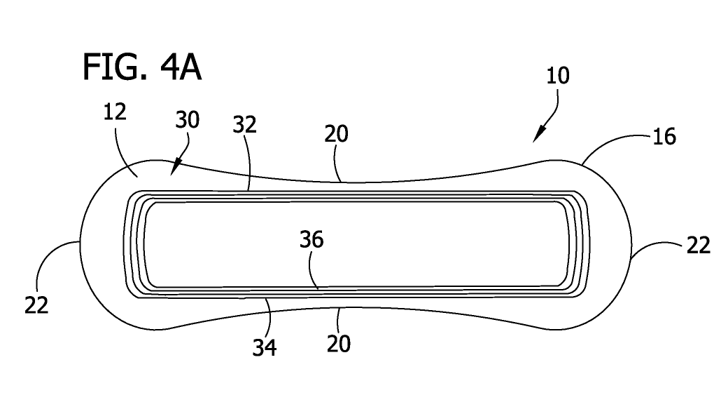

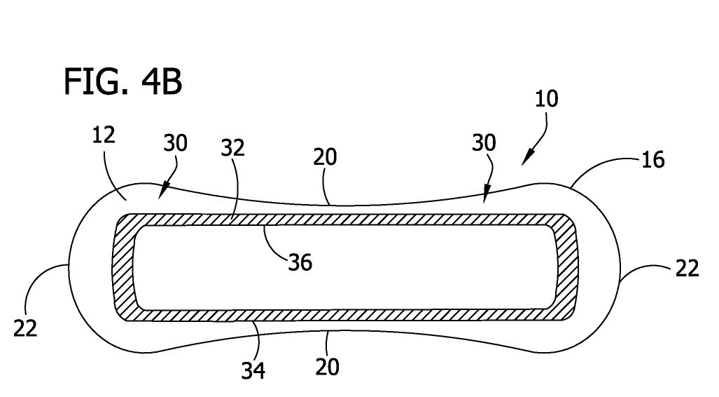

FIG. 4 illustrates an alternate embodiment wherein the band 30 is defined generally around a complete periphery of the article 10. As mentioned, although the focus of leakage from such articles 10 is generally along the lateral sides 20 of the article, leakage can also occur at the longitudinal ends 22. The embodiment of FIG. 4 may address this problem. It should also be appreciated that, although the band 30 is illustrated in FIG. 4 as having a laterally outboard deposit 34 spaced from the peripheral sealed edge 16 of the article 10, the deposits of pre-use foam composition 32 may just as well extend laterally outboard to the sealed edge 16.

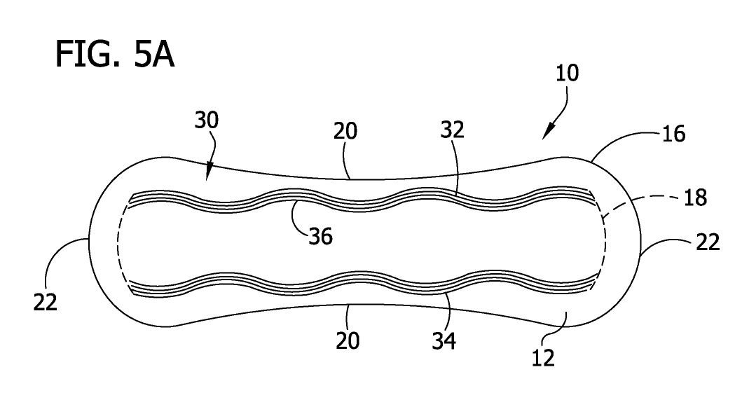

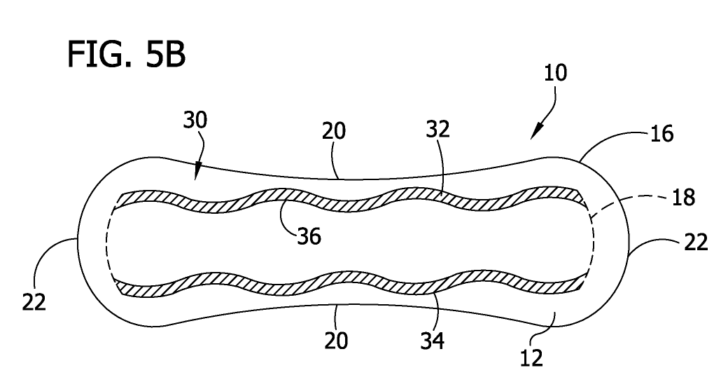

FIG. 5 illustrates an alternate embodiment wherein the bands 30 are defined by generally parallel and sinusoidal or wave-like deposits 32. The deposits 32 do not extend in this embodiment to the longitudinal ends 22 of the article 10, as compared to the embodiment of, for example, FIGS. 1 and 3.

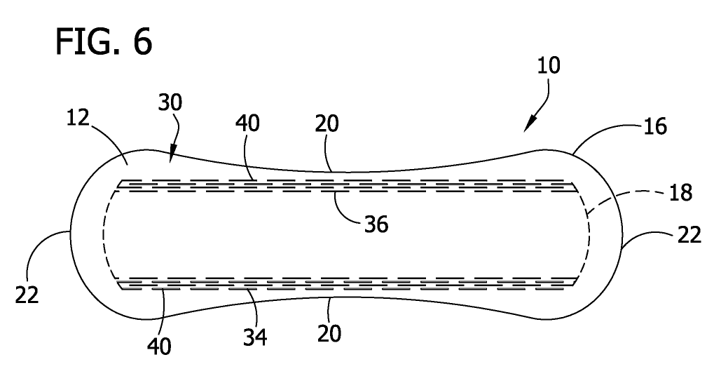

FIG. 6 illustrates an embodiment wherein the bands 30 are defined by discontinuous deposits of the pre-use foam composition. In this particular embodiment, the band 30 is defined by parallel dashed lines 40. It may be desired that the dashes 40 of the different individual parallel lines be interspaced such that a tortuous path is defined between the laterally inboard deposits 36 and laterally outboard deposits 34. In this manner, body exudates migrating laterally outboard are not provided with a clear unimpeded path to the lateral sides 20 of the article 10, but are caused to change directions numerous times before reaching the edges 16 of the article.

FIG. 7 illustrates an alternative embodiment of a discontinuous pattern for the bands 30. In this embodiment, the deposits are defined by individual drop-like deposits 38 on the topsheet 12.

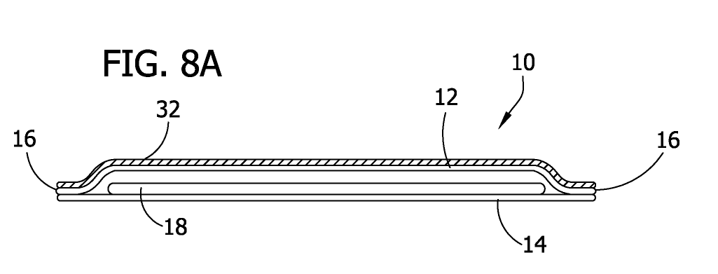

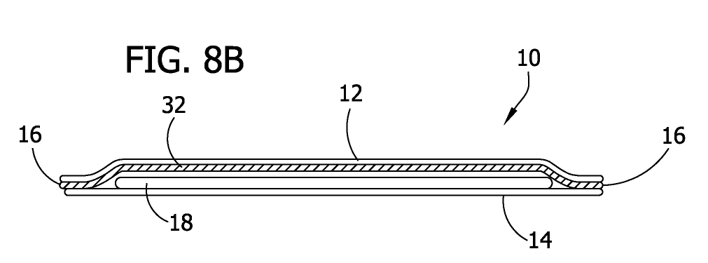

FIGS. 8A and 8B illustrate alternative embodiments, wherein a deposit of the pre-use foam composition 32 is uniformly distributed over the entire surface of one side of the topsheet 12 of an absorbent article 10. The pre-use foam composition deposit 32 may be provided on the body-facing surface of the topsheet 12, as shown in FIG. 8A, or may be deposited on the inner surface (facing the absorbent core 18) of the topsheet 12, as shown in FIG. 8B. Although illustrated in FIGS. 8A and 8B as a single layer deposit of pre-use foam composition 32, it is to be understood that components of the pre-use foam composition may be applied to the body-facing surface and/or the inner surface of the topsheet 12 as discrete layers of individual components, such as a layer of gas providing agent, a layer of foam forming agent, etc., as discussed above.

It should be appreciated that the pre-use foam composition may be deposited into or onto the absorbent articles in numerous configurations, and that the embodiments illustrated in the figures are for exemplary purposes only.

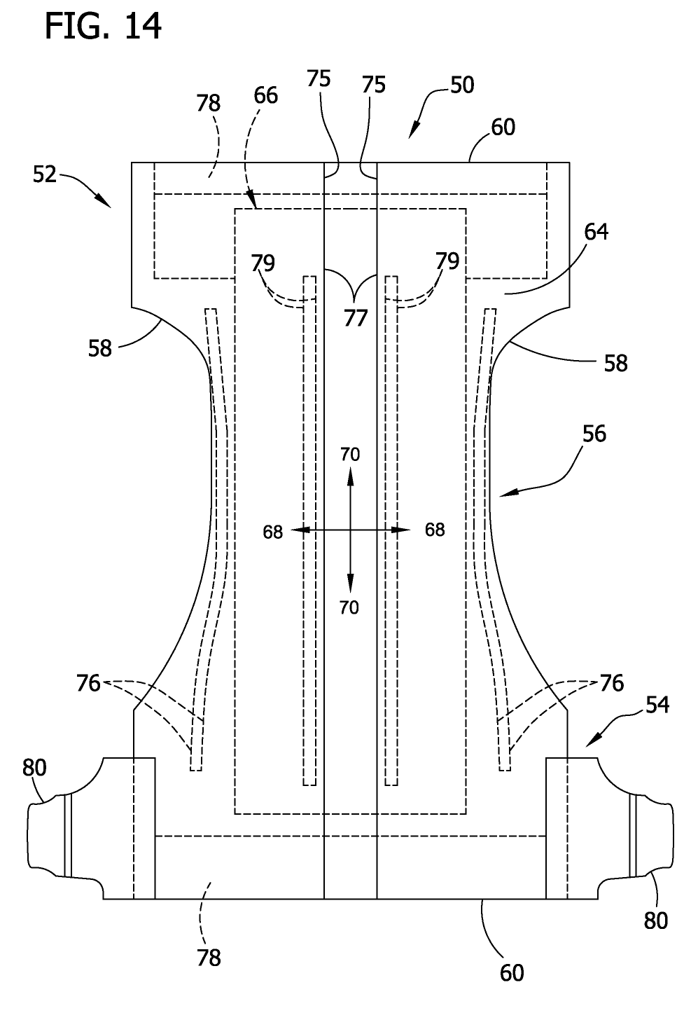

In certain embodiments, the foam barrier/interceptor structures of the present disclosure may be used in combination with other leakage protection features such as, for example, containment flaps or other pre-formed liquid impermeable barrier walls or cuffs, wings, elasticized leg flaps or side gathers, and the like. In one particular embodiment, the pre-use foam composition may be disposed on the topsheet or bodyside liner underneath a containment flap, such as the containment flaps illustrated in FIG. 14, described elsewhere herein, or another such sheet of material that comprises an unattached end free from connection with the topsheet or bodyside liner of the absorbent article. When the pre-use foam composition is contacted by the triggering agent, a foam barrier/interceptor structure forms, raising the unattached end of the containment flap or other such sheet into a generally upright position. This configuration advantageously provides a second barrier structure in addition to the foam barrier/interceptor structure formed by contact of the triggering agent with the pre-use foam composition.

Additionally, the raised containment flap or other such material may act to support the foam barrier/interceptor structure providing additional stability to the foam structure.

### Absorbent Articles

The present disclosure relates to any manner of absorbent article, such as diapers, training pants, swim pants, incontinence articles, feminine care articles such as sanitary napkins and panty liners, health care absorbent articles, and the like. The construction and materials used in conventional absorbent articles vary widely and are well known to those of skill in the art. The invention has particular usefulness for feminine care articles and, for purposes of illustration and description only, embodiments of feminine care articles according to the disclosure, in particular sanitary napkins, are referenced herein. However, it should be appreciated that the disclosure is in no way limited to sanitary napkins in particular, or to feminine care articles in general.

Referring to FIGS. 1 to 11 in general, an absorbent article 10 according to the disclosure includes a generally liquid permeable topsheet 12, an absorbent core 18 disposed beneath the topsheet, and optionally a generally liquid impermeable outer cover 14 (also referred to herein as a backsheet) disposed beneath the absorbent core 18. It should be understood that in certain embodiments, a single integrated material could act as a topsheet, an absorbent core, and an outer cover, or some combination thereof. The topsheet 12 and outer cover 14 may be sealed together at their peripheral edges utilizing known techniques, such as, for example, gluing, crimping, hot-sealing or the like, the sealed edges defining an overall sealed peripheral edge 16 of the article 10. The article 10 may take on various shapes, but will generally have opposite lateral sides 20 and longitudinal ends 22. Various geometries of absorbent articles, including feminine care articles, are well known to those skilled in the art, and all such embodiments are within the scope and spirit of the invention.

The topsheet 12 may include a layer constructed of any operative material, and may be a composite material. For example, the topsheet 12 can include a woven fabric, a nonwoven fabric, a polymer film, a film-fabric laminate or the like, as well as combinations thereof. As used herein, the term “nonwoven” refers to a fabric web that has a structure of individual fibers or filaments which are interlaid, but not in an identifiable repeating manner. Examples of a nonwoven fabric include spunbond fabric, meltblown fabric, coform fabric, a carded web, a bonded-carded-web, a bicomponent spunbond fabric or the like as well as combinations thereof.

For example, the topsheet 12 can include a woven fabric, a nonwoven fabric, a polymeric film that has been configured to be operatively liquid-permeable, or the like, as well as combinations thereof. Other examples of suitable materials for constructing the topsheet 12 can include rayon, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs, net materials, and the like, as well as combinations thereof.

A more particular example of a suitable topsheet 12 material can include a bonded-carded-web composed of polypropylene and polyethylene, such as has been used as a cover stock for KOTEX brand pantiliners, and has been obtainable from Vliesstoffwerk Christian Heinrich Sandler GmbH & Co. KG, a business having an address at Postfach 1144, D95120 Schwarzenbach/Saale, Germany. Other examples of suitable materials are composite materials of a polymer and a nonwoven fabric material. The composite materials are typically in the form of integral sheets generally formed by the extrusion of a polymer onto a web of spunbond material. As used herein, the terms “spunbond” or “spunbonded fiber” refer to fibers which are formed by extruding filaments of molten thermoplastic material from a plurality of fine, usually circular, capillaries of a spinneret, and then rapidly reducing the diameter of the extruded filaments.

In a desired arrangement, the topsheet 12 can be configured to be operatively liquid-permeable with regard to the liquids that the absorbent article 10 is intended to absorb or otherwise handle. The operative liquid-permeability may, for example be provided by a plurality of pores, perforations, apertures or other openings, as well as combinations thereof, that are present or formed in the topsheet. The apertures or other openings can help increase the rate at which bodily liquids can move through the thickness of the topsheet 12 and penetrate into the other components of the absorbent article 10 (e.g., the absorbent core 18). The selected arrangement of liquid-permeability is desirably present at least on an operative portion of the topsheet 12 that is appointed for placement on the body-side of the napkin 10.

The topsheet 12 can provide comfort and conformability, and can function to direct bodily exudates away from the body and toward the absorbent core 18. In a desired feature, the topsheet 12 can be configured to retain little or no liquid in its structure, and can be configured to provide a relatively comfortable and non-irritating bodyside surface (broadly, an “engagement surface”) next to the tissue layer (i.e., skin) of a wearer. The topsheet 12 can be constructed of any material easily penetrated by bodily fluids that contact the surface of the topsheet.

The topsheet 12 can also have at least a portion of its bodyside surface treated with a surfactant to render the topsheet more hydrophilic. The surfactant can permit arriving bodily liquids to more readily penetrate the topsheet 12. The surfactant may also diminish the likelihood that the arriving bodily fluids, such as menstrual fluid, will flow off the topsheet 12 rather than penetrate through the topsheet into other components of the absorbent article 10 (e.g., the absorbent core 18). In a particular configuration, the surfactant can be substantially evenly distributed across at least a portion of the upper, bodyside surface of the topsheet 12 that overlays the upper, bodyside surface of the absorbent core 18.

The topsheet 12 may be maintained in secured relation with the absorbent core 18 by bonding all or a portion of the adjacent surfaces to one another. A variety of bonding articles known to one of skill in the art may be utilized to achieve any such secured relation. Examples of such articles include, but are not limited to, the application of adhesives in a variety of patterns between the two adjoining surfaces, entangling at least portions of the adjacent surface of the absorbent with portions of the adjacent surface of the topsheet 12, or fusing at least portions of the adjacent surface of the topsheet to portions of the adjacent surface of the absorbent core 18.

The topsheet 12 typically extends over the upper, bodyside surface of the absorbent core 18, but can alternatively extend around the absorbent article 10 to partially or entirely, surround or enclose the absorbent core 18. Alternatively, the topsheet 12 and the outer cover 14 can have peripheral edges 16 that extend outwardly beyond the terminal, peripheral edges of the absorbent core 18, and the extending margins can be joined together to partially or entirely, surround or enclose the absorbent core.

The backsheet 14 may include a layer constructed of any operative material, and may or may not have a selected level of liquid-permeability or liquid-impermeability, as desired. In a particular configuration, the backsheet 14 may be configured to provide an operatively liquid-impermeable backsheet structure. The backsheet 14 may, for example, include a polymeric film, a woven fabric, a nonwoven fabric or the like, as well as combinations or composites thereof. For example, the backsheet 14 may include a polymer film laminated to a woven or nonwoven fabric. In a particular feature, the polymer film can be composed of polyethylene, polypropylene, polyester or the like, as well as combinations thereof. Additionally, the polymer film may be micro-embossed, have a printed design, and/or may be at least partially colored. Desirably, the backsheet 14 can operatively permit a sufficient passage of air and moisture vapor out of the absorbent article 10, particularly out of an absorbent core 18 while blocking the passage of bodily exudates. An example of a suitable backsheet 14 can include a breathable, microporous film, such as a HANJIN Breathable Baffle available from Hanjin Printing, Hanjin P&C Company Limited, a business having offices located in Sahvon-li.Jungan-mvu.Kongiu-City, Chung cheong nam-do, Republic of South Korea. The backsheet 14 material is a breathable film, which is white in color, dimple embossed and contains: 47.78% calcium carbonate, 2.22% TiO₂, and 50% polyethylene.

Bicomponent films or other multi-component films can also be used as backsheet 14 material, as well as woven and/or nonwoven fabrics which have been treated to render them operatively liquid-impermeable. Another suitable backsheet 14 material can include closed cell polyolefin foam. For example, closed cell polyethylene foam may be employed. Still another example of a backsheet 14 material would be a material that is similar to a polyethylene film which is used on commercially sold KOTEX brand pantiliners, and is obtainable from Pliant Corporation, a business having offices located in Schaumburg, Ill., USA.

The structure of the absorbent core 18 can be operatively configured to provide a desired level of absorbency or storage capacity. More particularly, the absorbent core can be configured to hold a liquid, such as urine, menses, complex liquid or the like, as well as combinations thereof. The absorbent core 18 can include a matrix of absorbent fibers and/or absorbent particulate material, and the absorbent fiber can include natural and/or synthetic fiber. Additionally, the absorbent core 18 may include one or more components that can modify menses or intermenstrual liquid.

The absorbent core 18 may also include superabsorbent material. Superabsorbent materials are known to those skilled in the art, and may be in any operative form, such as particulate form. Generally stated, the superabsorbent material can be a water-swellable, generally water-insoluble, hydrogel-forming polymeric absorbent material, which is capable of absorbing at least about 20, desirably about 30, and possibly about 60 times or more its weight in physiological saline (e.g. saline with 0.9 wt % NaCl). The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof.

The hydrogel-forming polymers are preferably lightly crosslinked to render the material substantially water insoluble. Crosslinking may, for example, be by irradiation or covalent, ionic, Van der Waals, or hydrogen bonding. Suitable materials are available from various commercial vendors such as The Dow Chemical Company and Stockhausen, Inc. The superabsorbent material may desirably be included in an appointed storage or retention portion of the absorbent system, and may optionally be employed in other components or portions of the absorbent article 10.

The absorbent core 18 can be arranged in any operative shape and/or design. For example, the absorbent core 18 may comprise a composite structure (not shown) having a selected plurality of strata or layers or a unitary structure. Moreover, the material of the absorbent core 18 can be selected and configured to provide desired liquid-intake properties to quickly absorb and pull liquid away from the body. Accordingly, the absorbent core 18 can provide the function of liquid intake and can also provide the functions of liquid distribution, spreading, liquid retention, and shape maintenance. The absorbent core 18 may include natural fibers, synthetic fibers, superabsorbent materials, a woven fabric; a nonwoven fabric; a wet-laid fibrous web; a substantially unbonded airlaid fibrous web; an operatively bonded, stabilized-airlaid fibrous web; or the like, as well as combinations thereof. Additionally, the absorbent core 18 may include one or more components that can modify menses or intermenstrual liquid.

In a particular arrangement, the absorbent core 18 can be a thermally-bonded, stabilized airlaid fibrous web available from Concert Fabrication (Concert code 225.1021), a business having offices located in Gatineaux, Quebec, Canada (e.g. Concert code 225.1021). The absorbent core 18 may also be a similar, stabilized airlaid fibrous web available from Buckeye Technologies, Inc., a business having offices located in Memphis, Tenn., U.S.A.

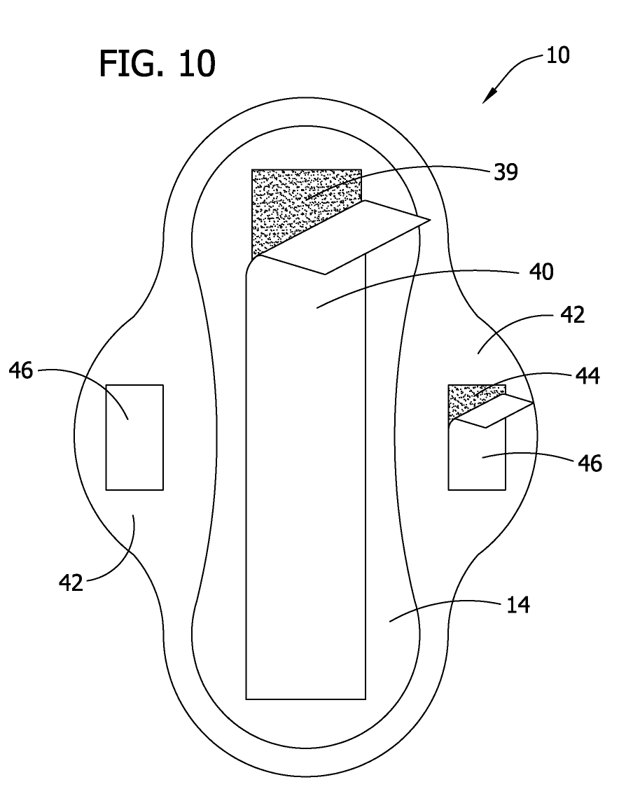

Additionally, a garment adhesive 39, such as the illustrated strip regions shown in FIG. 10, may be distributed onto the garment-side surface of the absorbent article 10 to help secure the article to the undergarment (not shown). Typically, the garment adhesive 39 can be distributed over the garment-side surface of the outer cover 14, and one or more layers or sheets of release material 40 can be removably placed over the garment adhesive 39 during storage prior to use.

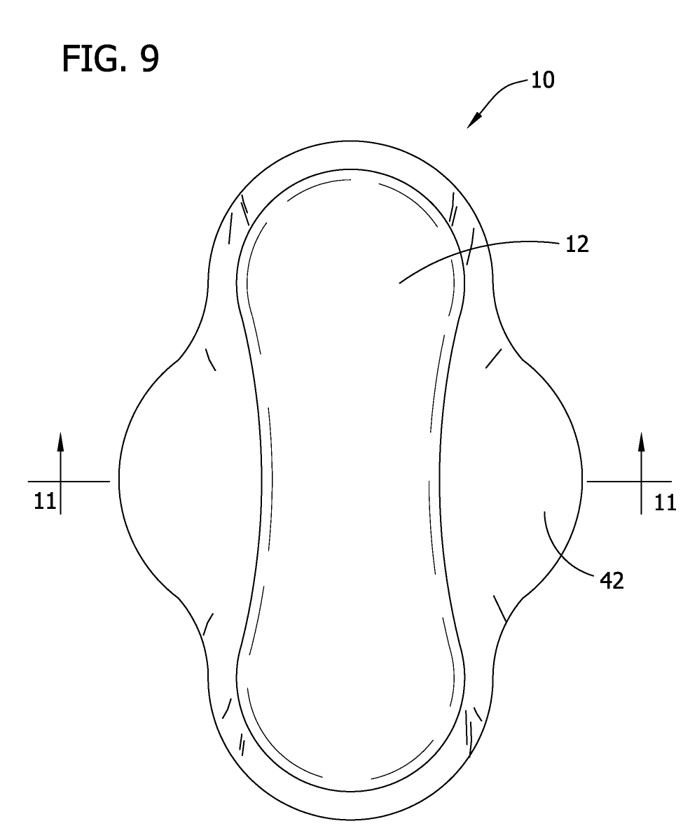

As illustrated in FIGS. 9-11, the absorbent article 10 can include a system of wing portions 42 which can be integrally connected to appointed sections of the absorbent article. After placing the article 10 in the undergarment, the wings 42 can be operatively wrapped and secured around the side edges of the undergarment to help hold the napkin in place as is known in the art. The wing portions 42 can be separately provided members that are subsequently attached or otherwise operatively joined to intermediate portions of the article 10.

In other configurations, the wing portions 42 can be unitarily formed with one or more components of the absorbent article 10. Either or both wing portions 42 may be formed from a corresponding, operative extension of the material employed to form the topsheet 12. Alternatively, either or both wing portions 42 may be formed from a corresponding, operative extension of the material employed to form the outer cover 14, or formed from a corresponding, operative combination of the topsheet 12 and outer cover 14 materials.

The wing portions 42 can have any operative construction, and can include a layer of any operative material. Additionally, each wing portion 42 can comprise a composite material. For example, the wing portions 42 may include a spunbond fabric material, a bi-component spunbond material, a necked spunbond material, a neck-stretched-bonded-laminate (NBL) material, a meltblown fabric material, a bonded carded web, a thermal bonded carded web, a through-air bonded carded web or the like, as well as combinations thereof.

Each wing portion 42 can be joined to its corresponding side region of the absorbent article 10 in any operative manner. For example, the wing portion can be joined to the topsheet 12, the outer cover 14 or another article 10 component, as well as any combination thereof. The wing portion 42 can be attached with hotmelt adhesive, but any other operative adhesive or attachment mechanism may alternatively be employed.

In another feature, each wing portion 42, or any desired combination of the employed wing portion, can include a panel-fastener component 44 which is operatively joined to an appointed engagement surface of its associated wing. The panel-fastener can be configured to operatively attach to the wearer's undergarment and/or to any appointed, landing-zone portion of the absorbent article 10. For example, the panel-fastener 44 can include a system of interengaging mechanical fasteners, a system of adhesive fasteners, a system of cohesive fasteners or the like, as well as combinations thereof.

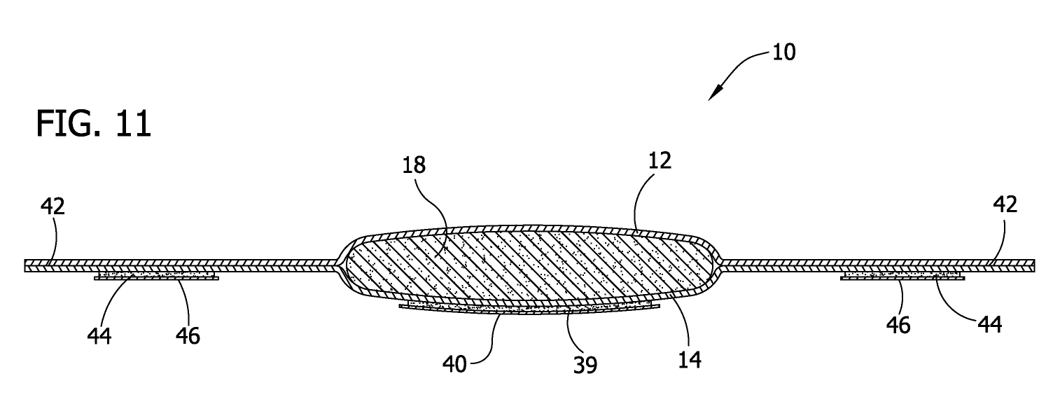

With reference to FIGS. 10 and 11, for example, either or both wing portions 42 can include a panel-fastener system 44 that alternatively incorporates an operative garment adhesive. The garment adhesive may be a solvent-base adhesive, a hotmelt adhesive, a pressure-sensitive adhesive, or the like, as well as combinations thereof. Each section of garment adhesive may be covered with a removable release material 46.

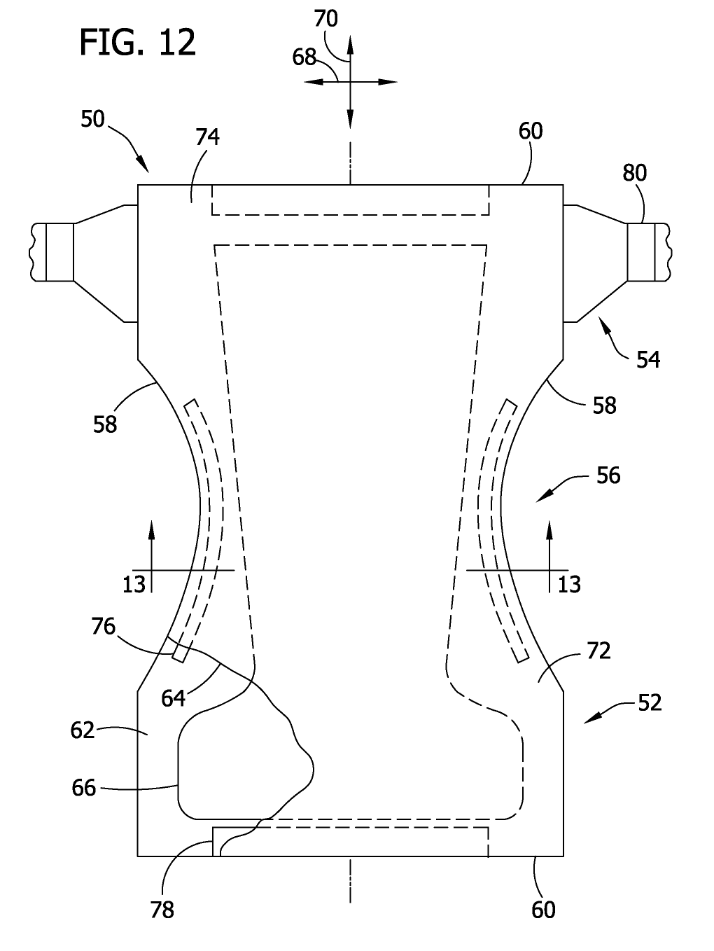

As discussed above, the present disclosure is also applicable to other absorbent articles, such as disposable diapers. With reference now to FIGS. 12-14, a disposable diaper 50, generally defines a front waist section 52, a rear waist section 54, an intermediate section 56 which interconnects the front and rear waist section, a pair of laterally opposed side edges 58, and a pair of longitudinally opposed end edges 60. The front and rear waist sections include the general portions of the article which are constructed to extend substantially over the wearer's front and rear abdominal regions, respectively, during use. The intermediate section of the article includes the general portion of the article, which is constructed to extend through the wearer's crotch region between the legs. The opposed side edges 58 define leg openings for the diaper and generally are curvilinear or contoured to more closely fit the legs of the wearer. The opposed end edges 60 define a waist opening for the diaper 50 and typically are straight but may also be curvilinear.

FIG. 12 is a representative plan view of the diaper 50 in a flat, non-contracted state. Portions of the structure are partially cut away to more clearly show the interior construction of the diaper 50, and the surface of the diaper which contacts the wearer is facing the viewer. The diaper 50 includes a substantially liquid impermeable outer cover (i.e., backsheet) 62, a porous, liquid permeable bodyside liner (i.e., topsheet) 64 positioned in facing relation with the outer cover 62, and an absorbent body 66, such as an absorbent pad, which is located between the outer cover and the bodyside liner. The diaper 50 also defines a lateral direction 68 and a longitudinal direction 70. Marginal portions of the diaper 50, such as marginal sections of the outer cover 62, may extend past the terminal edges of the absorbent body 66. In the illustrated embodiment, for example, the outer cover 62 extends outwardly beyond the terminal marginal edges of the absorbent body 66 to form side margins 72 and end margins 74 of the diaper 50. The bodyside liner 64 is generally coextensive with the outer cover 62, but may optionally cover an area which is larger or smaller than the area of the outer cover 62, as desired.

To provide improved fit and to help reduce leakage of body exudates from the diaper 50, the side margins 72 and end margins 74 of the diaper may be elasticized with suitable elastic members, such as leg elastic members 76 and waist elastic members 78. For example, the leg elastic members 76 may include single or multiple strands of elastic or elastomeric composites which are constructed to operably gather and shirr the side margins 72 of the diaper 50 to provide elasticized leg bands which can closely fit around the legs of the wearer to reduce leakage and provide improved comfort and appearance. Similarly, the waist elastic members 78 can be employed to elasticize the end margins 74 of the diaper 50 to provide elasticized waistbands. The waist elastics are configured to operably gather and shirr the waistband sections to provide a resilient, comfortably close fit around the waist of the wearer.

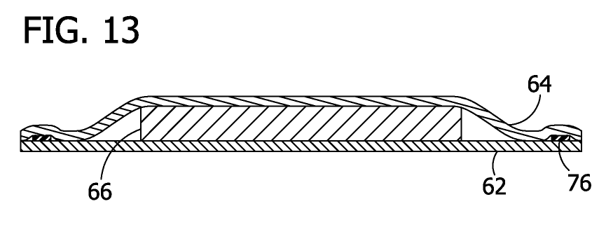

The elastic members 76 and 78 are secured to the diaper 50 in an elastically contractible condition so that in a normal under strain configuration, the elastic members effectively contract against the diaper 50. For example, the elastic members 76 and 78 may be elongated and secured to the diaper 50 while the diaper is in an uncontracted condition. In FIGS. 12 and 13, the elastic members 76 and 78 are illustrated in their uncontracted, stretched condition for the purpose of clarity.

The diaper 50 may also comprises a pair of elasticized, longitudinally extending containment flaps 75 configured to provide a barrier to the lateral flow of body exudates, as illustrated in FIG. 14. The containment flaps 75 are located generally adjacent laterally opposed side edges 58 of the diaper 50 and, when the diaper is laid flat as shown in FIG. 14, extend inward toward the longitudinal direction 70 of the diaper. Each containment flap 75 typically has a free, or unattached end 77 free from connection with the bodyside liner 64 and other components of the diaper 50. Elastic strands 79 disposed within the flaps 75 adjacent the unattached ends thereof urge the flaps toward an upright, perpendicular configuration in at least the intermediate section 56 of the diaper 50 to form a seal against the wearer's body when the diaper is worn. The containment flaps 75 may extend longitudinally the entire length of the diaper 50 or they may extend only partially along the length of the diaper. When the containment flaps 75 are shorter in length than the diaper 50, the flaps can be selectively positioned anywhere between the side edges 58 of the diaper 50 in the intermediate region 56. In a particular aspect of the invention, the containment flaps 75 extend the entire length of the diaper 50 to better contain the body exudates.

Such containment flaps 75 are generally well known to those skilled in the art and therefore will not be further described herein except to the extent necessary to describe the present invention. As an example, suitable constructions and arrangements for containment flaps 75 are described in U.S. Pat. No. 4,704,116 issued Nov. 3, 1987, to K. Enloe, the disclosure of which is hereby incorporated by reference. The diaper 50 may also incorporate other containment components in addition to or instead of the containment flaps 75. For example, while not shown in the drawings, other suitable containment components may include, but are not limited to foam dams in the front, back and/or crotch regions, and the like.

Alternatively or in addition, the diaper 50 may include a pair of separate, elasticized and gathered leg gussets (not shown) or combination leg gussets/containment flaps (not shown) which are attached to the diaper along the side margins 72 in at least the intermediate section 56 of the diaper 50 to provide elasticized leg cuffs. Such gussets or combination gussets/containment flaps may be configured to extend beyond and bridge across the respective concave portion of the side margins 72.

The diaper 50, as representatively illustrated in FIGS. 12 and 13, may further include a pair of fasteners 80 employed to secure the diaper 50 about the waist of a wearer. Suitable fasteners 80 include hook-and-loop type fasteners, adhesive tape fasteners, buttons, pins, snaps, mushroom-and-loop fasteners, and the like. A cooperating side panel member can be associated with each fastener and may be constructed to be nonelasticized, or to be elastically stretchable at least along the lateral direction 68 of diaper 50.

The diaper may further include a surge management layer (not shown) positioned between the bodyside liner 64 and the absorbent body 66 which is configured to efficiently hold and distribute liquid exudates to the absorbent body 66. The surge management layer can prevent the liquid exudates from pooling and collecting on the portion of the diaper positioned against the wearer's skin, thereby reducing the level of skin hydration. Suitable constructions and arrangements of surge management layers are well known to those skilled in the art. Other suitable diaper components may also be incorporated on absorbent articles described herein.

The diaper 50 may be of various suitable shapes. For example, the diaper may have an overall rectangular shape, T-shape, or an approximately hour-glass shape. In the shown embodiment, the diaper 50 is I-shaped. Examples of diaper configurations suitable for use in connection with the instant application and other diaper components suitable for use on diaper 50 are described in U.S. Pat. No. 4,798,603 issued Jan. 17, 1989 to Meyer et al.; U.S. Pat. No. 5,176,668 issued Jan. 5, 1993, to Bernardin; U.S. Pat. No. 5,176,672 issued Jan. 5, 1993 to Bruemmer et al.; U.S. Pat. No. 5,192,606 issued Mar. 9, 1993 to Proxmire et al.; and U.S. Pat. No. 5,509,915 issued Apr. 23, 1996 to Hanson et al., the disclosures of which are hereby incorporated by reference. The various aspects and configurations of the invention can provide distinctive combinations of softness, body conformity, reduced red-marking of the wearer's skin, reduced hydration, and improved containment of body exudates.

The various components of the diaper 50 are integrally assembled together employing various types of suitable attachment means, such as adhesive, sonic bonds, thermal bonds, or combinations thereof. In the shown embodiment, for example, the bodyside liner 64 and the outer cover 62 are assembled to each other and to the absorbent body 66 with adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Similarly, other diaper components, such as the elastic members 76 and 78 and the fasteners 80, may be assembled into the diaper 50 by employing the above-identified attachment mechanisms. Examples of suitable materials that may be used to form the bodyside liner 64, outer cover 62, and absorbent body 66 are known in the art and described in, for example, U.S. Patent App. No. 2006/0036222, herein incorporated by reference.

Having described the disclosure in detail, it will be apparent that modifications and variations are possible without departing from the scope of the disclosure defined in the appended claims.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above products products and methods without departing from the scope of the disclosure, it is intended that all matter contained in the above description and shown in the accompanying drawings shall be interpreted as illustrative and not in a limiting sense.

What is claimed is:

1. An absorbent article comprising:
a topsheet;
an absorbent core disposed beneath the topsheet; and
a system for generating a stable foam structure on a body facing surface of the topsheet, the system comprising a pre-use foam composition comprising a gas providing agent and a foam forming agent,
wherein the pre-use foam composition is disposed on the absorbent article in a manner such that when the gas providing agent is contacted with a triggering agent, the gas providing agent releases a gas that combines with the foam forming agent to form the stable foam structure, wherein the stable foam structure acts as a fluid barrier against the flow of a body exudate across the barrier, as an interceptor, or as a fluid barrier and an interceptor.

2. The absorbent article of claim 1 wherein the stable foam structure is a wet liquid foam.

3. The absorbent article of claim 2 wherein the wet liquid foam has a viscosity of at least about 75,000 mPa*s.

4. The absorbent article of claim 3 wherein the wet liquid foam has a yield stress of about 30,000 dyne/cm² or less.

5. The absorbent article of claim 1 wherein the pre-use foam composition is disposed on the body facing surface of the topsheet.

6. The absorbent article of claim 1 wherein the pre-use foam composition is deposited on the topsheet in longitudinally extending bands on opposite sides of a longitudinal centerline of the absorbent article.

7. The absorbent article of claim 1 wherein the gas providing agent releases the gas through an effervescent reaction.

8. The absorbent article of claim 1 wherein the gas providing agent comprises an acid and a base.

9. The absorbent article of claim 8 wherein the acid is citric acid and the base is sodium bicarbonate.

10. The absorbent article of claim 1 wherein the gas providing agent comprises a liquid-soluble solid material containing a gas entrapped within cells located in the solid material.

11. The absorbent article of claim 10 wherein the liquid-soluble solid material is selected from the group consisting of sugars, salts, alkali halides, alkaline earth metal halides, and combinations thereof.

12. The absorbent article of claim 1 wherein the gas providing agent is encapsulated.

13. The absorbent article of claim 1 wherein the gas is selected from the group consisting of carbon dioxide, air, nitrogen, oxygen, argon, helium, neon, krypton, xenon, radon, and combinations thereof.

14. The absorbent article of claim 1 wherein the foam forming agent is selected from the group consisting of viscoelastic surfactants, lathering surfactants, emollients, and combinations thereof.

15. The absorbent article of claim 1 wherein the foam forming agent is encapsulated.

16. The absorbent article of claim 1 wherein the triggering agent is selected from the group consisting of body exudates, heat, pressure, and combinations thereof.

17. The absorbent article of claim 1 wherein the pre-use foam composition further comprises a foam stabilizing agent.

18. The absorbent article of claim 17 wherein the foam stabilizing agent is an acyl lactylate.

19. The absorbent article of claim 1 wherein the barrier structure is continuously formed during use of the absorbent article.

20. The absorbent article of claim 1 wherein the pre-use foam composition further comprises a component selected from the group consisting of skin feel enhancing agents, adherence agents, anti-adherence agents, trigger control agents, water, and combinations thereof.

21. The absorbent article of claim 20 wherein the pre-use foam composition further comprises a skin feel enhancing agent.

22. The absorbent article of claim 21 wherein the pre-use foam composition comprises about 30% of the gas providing agent, about 30% of the foam forming agent, about 30% of the foam stabilizing agent, and about 10% of the skin feel enhancing agent.

23. The absorbent article of claim 1 wherein the absorbent article is selected from the group consisting of diapers, training pants, swim pants, incontinence articles, feminine care articles, and health care absorbent articles.

24. The absorbent article of claim 1 further comprising a leakage protection feature selected from the group consisting of wings, containment flaps, liquid impermeable walls or cuffs, elasticized leg flaps, and combinations thereof.

25. The absorbent article of claim 24 wherein the absorbent article further comprises containment flaps, the pre-use foam composition being disposed on the topsheet underneath the containment flaps.

26. A composition for generating a stable foam structure, the composition comprising a gas providing agent and a foam forming agent, wherein the gas providing agent releases a gas that combines with the foam forming agent to form the stable foam structure when the composition is contacted with a triggering agent.
